(19) United States
(12) Patent Application Publication
(10) Pub. No.: US 2018/0052158 A1
(43) Pub. Date: Feb. 22, 2018

(54) METHOD AND KIT FOR EXAMINING COMPLEMENT SYSTEM

(71) Applicant: National University Corporation Asahikawa Medical University, Asahikawa-shi (JP)

(72) Inventors: Nobutaka Wakamiya, Asahikawa-shi (JP); Katsuki Ohtani, Asahikawa-shi (JP)

(73) Assignee: National University Corporation Asahikawa Medical University, Asahikawa-shi (JP)

(21) Appl. No.: 15/672,661

(22) Filed: Aug. 9, 2017

(30) Foreign Application Priority Data
Aug. 16, 2016 (JP) 2016-159453

(51) Int. Cl. G01N 33/557; G01N 33/50

(52) U.S. Cl. CPC G01N 33/557; G01N 2333/4716; G01N 33/502

(57) ABSTRACT

A method for examining a complement system of a subject, including: a reaction step of letting a scavenger receptor or a functional equivalent thereof, a pentraxin family protein or a functional equivalent thereof and a blood sample collected from the subject coexist in vitro; and a detection step of detecting complement activation amplifying reaction and/or complement activation late-phase reaction induced by interaction between the scavenger receptor or the functional equivalent thereof and the pentraxin family protein or the functional equivalent thereof from a reactant after the reaction step. Also provided are a kit for examining a complement system, and a method for testing a test substance for the ability to activate or suppress a complement system.

### CROSS-REFERENCE TO RELATED APPLICATIONS

This application claims priority to Japanese application No. 2016-159453, filed Aug. 16, 2016, the content of which is incorporated herein by reference in its entirety for all purposes.

### TECHNICAL FIELD

The present invention relates to a method for examining activation of the complement system and/or abnormality of the complement system of a subject, and a kit for the examination.

### BACKGROUND OF THE INVENTION

The complement system is a kind of innate immunity that is activated in response to invasion of a pathogen, and is composed of a large number of complement proteins that exist as membrane-bound proteins or soluble proteins in blood.

The complement system activating pathways are roughly classified into three pathways: a classical pathway that is specifically activated by antigen-antibody reaction against a pathogen, a secondary pathway that is not accompanied by a mechanism of recognizing a pathogen, and a lectin pathway that is activated by recognition of a sugar chain of a pathogen. Each pathway of complement activation and complement proteins forming each pathway are schematically shown in FIG. 1.

All of the three pathways of complement activation proceed via generation of C3b by degradation of C3 which is one of complement proteins (also referred to as C3 activation, or C3b deposition). C3 is a central mediating factor of inflammation, and is activated by a variety of causative factors of inflammation (typically infection).

In activation of the complement system, C3 activation is amplified by an amplification pathway where C3b binds to Bb which is a degradation product of B factor to form C3bBb, and this further promotes C3 activation. All of the three pathways of complement activation lead to formation of a terminal complement complex (TCC). TCC is a complex of C5b to C9, and is also referred to as C5b-9. C5b-9, as a membrane attack complex (MAC) has a function of lysing and removing pathogen cells.

While the complement system is originally a biological defense mechanism against invasion of pathogens, excessive activation of the complement system can impose severe tissue damage on an organism because MAC also causes lysis of cells other than pathogens, for example own cells of the organism.

The complement system is believed to be associated with various diseases such as autoimmune disease and inflammatory diseases. Examples of the diseases for which down regulation of the complement system (suppression of activation) has been suggested to be therapeutically effective include systemic lupus erythematosus and nephritis (Y. Wang et al., Proc. Natl. Acad. Sci.; 1996, 93:8563-8568), rheumatoid arthritis (Y. Wang et al., Proc. Natl. Acad. Sci., 1995; 92:8955-8959), myocardial infarction (J. W. Homeister et al., J. Immunol. 1993; 150:1055-1064), and reperfusion injury (E. A. Amsterdam et al., Am. J. Physiol. 1995; 268:H448-H457). In these diseases, it has been inferred that activation of the complement system causes or aggravates the respective symptoms.

Therefore, knowing whether or not the complement system is activated in a patient, and further knowing whether there is abnormality in the regulatory mechanism of the complement system make it possible to estimate the involvement of the complement system in the disease, and give clinically beneficial information for decision of a therapeutic strategy and selection of appropriate therapy.

One method for examining whether or not the complement system is activated is to use the ultimate cytolysis as an index (collective measuring method). A representative example is CH50 (Complement Hemolysis 50%), which is a method of using 50% hemolytic activity for sheep erythrocyte sensitized with hemolysis as an index.

The measurement result of CH50 is indicated as a complement titer. It is generally understood that activation of the complement system increases the hemolytic activity and increases the complement titer (high complement titer), but enhanced activation increases the consumption of complement proteins, and decreases the complement titer (low complement titer). Therefore, it is considered that low complement titer is clinically more important information than high complement titer. The complement titer tends to fall, for example, in immune complex diseases, chronic hepatic diseases, complement component deficiencies and the like.

While CH50 has been widely used as a method for measuring a complement titer, it has the problems including complicated operation in serial dilution of the test sample, occurrence of a measurement error caused by the serial dilution, and variation in the sensitivity of sheep erythrocytes among rots (sensitivity to the complement system varies every time erythrocytes are prepared). Further, since activation of the complement system is collectively measured, it is difficult to specify which complement protein has abnormality (lack of protein, excessive or deficient amount of protein, etc.). A method for examining the ability to activate the second pathway using 50% hemolytic activity of rabbit erythrocytes, which is called ACH50 also has the same problems as CH50.

As a collective measuring method different from CH50, a liposome method using liposome that is prepared to be susceptible to the membrane injury reaction by the complement activity in place of erythrocytes has been reported (see, for example, JP 1-155271 A). Although this method solves the problem of variation among rots by using liposome, it fails to solve the problem accompanied with dilution of a sample, and the problem of disability to specify the location of the abnormality.

### SUMMARY OF THE INVENTION

### Technical Problem

It is an object of the present invention to provide a more simple and reliable method for examining a complement system.

### Solution to Problem

The present inventors found that the interaction between a scavenger receptor and a pentraxin family protein is involved in activation of the complement system, and that the complement system can be examined by reproducing a binding between the scavenger receptor and the pentraxin family protein in vitro, and letting these coexist with a blood sample of a subject, and accomplished the following aspects of the invention.

(1) A method for examining a complement system of a subject, including:

a reaction step of letting a scavenger receptor or a functional equivalent thereof, a pentraxin family protein or a functional equivalent thereof and a blood sample collected from the subject coexist in vitro; and

a detection step of detecting complement activation amplifying reaction and/or complement activation late-phase reaction induced by interaction between the scavenger receptor or the functional equivalent thereof and the pentraxin family protein or the functional equivalent thereof from a reactant after the reaction step.

(2) The method according to (1), wherein in the detection step, complement activation early-phase reaction is further detected.
(3) The method according to (1), wherein the reaction step is a step of letting the scavenger receptor or the functional equivalent thereof existing on mammalian cells, the pentraxin family protein or the functional equivalent thereof and the blood sample collected from the subject coexist.
(4) The method according to (3), wherein the mammalian cells are human cells.
(5) The method according to (3), wherein the mammalian cells are human embryonic kidney-derived cells.
(6) The method according to (1), wherein the reaction step is a step of letting the scavenger receptor or the functional equivalent thereof immobilized on a support, the pentraxin family protein or the functional equivalent thereof and the blood sample collected from the subject coexist.
(7) The method according to (6), wherein the functional equivalent of the scavenger receptor is a peptide fragment containing an extracellular domain of the scavenger receptor.
(8) The method according to (1), wherein the scavenger receptor is CL-P1, SR-AI or LOX-1.
(9) The method according to (1), wherein the pentraxin family protein is C-reactive protein, serum amyloid P component or pentraxin 3.
(10) The method according to (1), wherein the detection of the complement activation amplifying reaction is conducted by detection of properdin and/or factor B or a degradation product thereof.
(11) The method according to (1), wherein the detection of the complement activation late-phase reaction is conducted by detection of C5b-9.
(12) The method according to (2), wherein the detection of the complement activation early-phase reaction is conducted by detection of a C3 degradation product.
(13) A kit for examining a complement system, including:

human cells expressing a scavenger receptor or a functional equivalent thereof; and

a pentraxin family protein or a functional equivalent thereof.

(14) A method for testing a test substance for the ability to activate or suppress a complement system, including:

a test substance reaction step of letting a scavenger receptor or a functional equivalent thereof, a pentraxin family protein or a functional equivalent thereof, a blood sample and a test substance coexist in vitro;

a control reaction step of letting a scavenger receptor or a functional equivalent thereof, a pentraxin family protein or a functional equivalent thereof and a blood sample coexist in vitro;

a detection step of detecting complement activation amplifying reaction and/or complement activation late-phase reaction induced by interaction between the scavenger receptor or the functional equivalent thereof and the pentraxin family protein or the functional equivalent thereof from a reactant after each reaction step; and

a determination step of determining that the test substance has the ability to activate the complement system when the complement activation amplifying reaction and/or the complement activation late-phase reaction detected from the reactant after the test substance reaction step is larger than the complement activation amplifying reaction and/or the complement activation late-phase reaction detected from the reactant after the control reaction step, and determining that the test substance has the ability to suppress the complement system when the complement activation amplifying reaction and/or the complement activation late-phase reaction detected from the reactant after the test substance reaction step is smaller than the complement activation amplifying reaction and/or the complement activation late-phase reaction detected from the reactant after the control reaction step.

### Advantageous Effects of Invention

Since the method and the kit according to one or more embodiments of the present invention do not require serial dilution of a blood sample, and do not use hemolysis of ovine erythrocytes as an index, they do not have the problems of complicated operation and measurement errors caused thereby, and variation among rots. The method and the kit of the present invention can detect both complement activation amplifying reaction and complement activation late-phase reaction, and further detection of the complement activation early-phase reaction in addition to the above two reactions enables collective evaluation of the complement system activation as well as specification of the complement protein where abnormality (lack of protein, excessive or deficient amount of protein, etc.) exists.

### BRIEF DESCRIPTION OF THE DRAWINGS

FIG. 1 is a schematic diagram showing pathways of complement activation, and complement proteins constituting respective pathways;

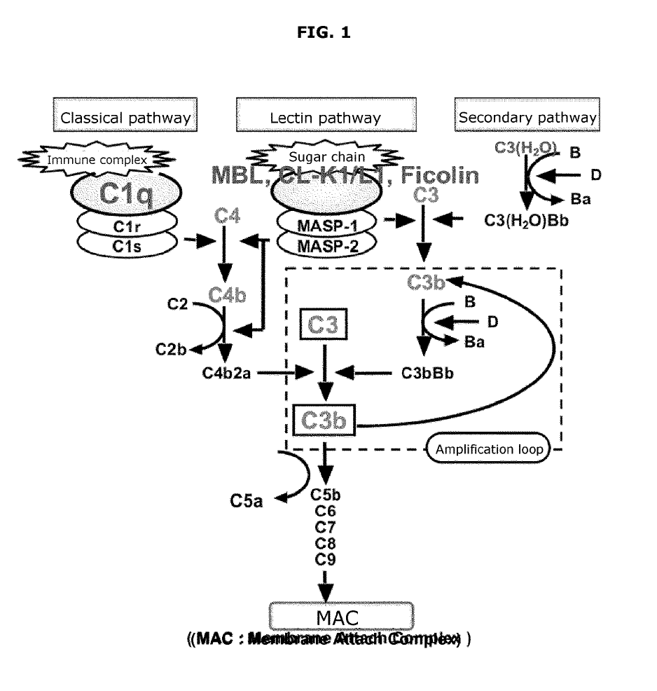

FIG. 2 shows graphs showing binding between CL-P1 extracellular domain, and C-reactive protein (CRP), serum amyloid P component (SAP) or pentraxin 3 (PTX3) in the ELISA system, FIG. 2A shows the result of CRP, FIG. 2B shows the result of SAP, and FIG. 2C shows the result of PTX3. The asterisk indicates a significant difference (p<0.0001) relative to BSA control, the double dagger indicates a significant difference (p<0.0001) of undenatured CRP, SAP or PTX3 relative to control;

FIG. 3 shows fluorescent micrographs (scale bar: 20 μm) of CHO/ldlA7 cells expressing CL-P1 or pcDNA3.1 control vector after incubation together with CRP, SAP or PTX3, in which CL-P1 and CRP, SAP or PTX3 are fluorescently detected;

FIG. 4 is a graph showing binding amounts of CRP, SAP or PTX3 versus CL-P1 expression amount, calculated from a mean fluorescent intensity of a fluorescent image of CHO/ldlA7 cells expressing CL-P1 or pcDNA3.1 control vector after incubation together with CRP, SAP or PTX3, in which CL-P1 and CRP, SAP or PTX3 are fluorescently detected;

FIG. 5 is a graph showing C3d deposition amounts generated by incubation of CL-P1 extracellular domain or BSA together with human complement serum in the presence or absence of CRP in the ELISA system;

FIG. 6 is a graph showing C3d deposition amounts generated by incubation of CL-P1 extracellular domain or BSA together with C1q depleted serum or C1q depleted serum supplemented with C1q in the presence or absence of CRP, SAP or PTX3 in the ELISA system;

FIG. 7 shows fluorescent micrographs (scale bar: 20 μm) of HEK293 cells expressing CL-P1 or pcDNA3.1 control vector after incubation together with human complement serum in the presence or absence of CRP, in which CL-P1 and C3d are fluorescently detected;

FIG. 8 is a graph showing C3d deposition amounts generated by incubation of HEK293 cells expressing CL-P1 or pcDNA3.1 control vector together with human complement serum in the presence or absence of CRP;

FIG. 9 shows fluorescent micrographs (scale bar: 20 μm) of HEK293 cells expressing CL-P1 or pcDNA3.1 control vector after incubation together with human complement serum in the presence or absence of CRP, in which C1q and C3d are fluorescently detected;

FIG. 10 is a graph showing C3d deposition amounts generated by incubation of HEK293 cells expressing CL-P1 or pcDNA3.1 control vector together with C1q depleted serum or C1q depleted serum supplemented with C1q in the presence or absence of CRP, SAP or PTX3;

FIG. 11 is a graph showing C3d deposition amounts generated by incubation of CL-P1 extracellular domain or BSA together with CFB depleted serum or CFB depleted serum supplemented with CFB in the presence or absence of CRP, SAP or PTX3 in the ELISA system;

FIG. 12 shows fluorescent micrographs (scale bar: 20 μm) of CL-P1 expressing HEK293 cells after incubation together with human complement serum in the presence or absence of CRP, SAP or PTX3, in which CL-P1 and CFB are fluorescently detected;

FIG. 13 is a graph showing C3d deposition amounts generated by incubation of HEK293 cells expressing CL-P1 or pcDNA3.1 control vector together with CFB depleted serum or CFB depleted serum supplemented with CFB in the presence or absence of CRP, SAP or PTX3;

FIG. 14 is a graph showing C3d deposition amounts generated by incubation of CL-P1 extracellular domain or BSA together with properdin depleted serum or properdin depleted serum supplemented with properdin in the presence or absence of CRP, SAP or PTX3 in the ELISA system;

FIG. 15 shows fluorescent micrographs (scale bar: 20 μm) of CL-P1 expressing HEK293 cells after incubation together with human complement serum in the presence or absence of CRP, SAP or PTX3, in which CL-P1 and properdin are fluorescently detected;

FIG. 16 is a graph showing C3d deposition amounts generated by incubation of HEK293 cells expressing CL-P1 or pcDNA3.1 control vector together with properdin depleted serum or properdin depleted serum supplemented with properdin in the presence or absence of CRP, SAP or PTX3;

FIG. 17 shows fluorescent micrographs (scale bar: 20 μm) of CL-P1 expressing HEK293 cells after incubation together with human complement serum in the presence or absence of CRP, SAP or PTX3, in which CL-P1 (FIG. 17A-C), C5b-9 (FIG. 17A), CFH (FIG. 17B) and C4BP (FIG. 17C) are fluorescently detected;

FIG. 18 is a graph showing C5b-9 deposition amounts formed by incubation of CL-P1 extracellular domain or BSA together with CFH depleted serum or CFH depleted serum supplemented with CFH in the presence or absence of CRP, SAP or PTX3 in the ELISA system;

FIG. 19 is a graph showing C5b-9 deposition amounts formed by incubation of CL-P1 extracellular domain or BSA together with C4BP depleted serum or C4BP depleted serum supplemented with C4BP in the presence or absence of CRP, SAP or PTX3 in the ELISA system;

FIG. 20 is a graph showing C5b-9 deposition amounts formed by incubation of CL-P1 extracellular domain or BSA together with CFI depleted serum or CFI depleted serum supplemented with CFI in the presence or absence of CRP, SAP or PTX3 in the ELISA system;

FIG. 21 is a graph showing C5b-9 deposition amounts formed by incubation of HEK293 cells expressing CL-P1 or pcDNA3.1 control vector together with CFH depleted serum or CFH depleted serum supplemented with CFH in the presence or absence of CRP, SAP or PTX3;

FIG. 22 is a graph showing C5b-9 deposition amounts formed by incubation of HEK293 cells expressing CL-P1 or pcDNA3.1 control vector together with C4BP depleted serum or C4BP depleted serum supplemented with C4BP in the presence or absence of CRP, SAP or PTX3;

FIG. 23 is a graph showing C5b-9 deposition amounts formed by incubation of HEK293 cells expressing CL-P1 or pcDNA3.1 control vector together with CFI depleted serum or CFI depleted serum supplemented with CFI in the presence or absence of CRP, SAP or PTX3;

FIG. 24 is a graph showing C5b-9 deposition amounts formed by incubation of HEK293 cells expressing CL-P1 or pcDNA3.1 control vector together with atypical hemolytic uremic syndrome (aHUS) patient serum or aHUS patient serum supplemented with CFH in the presence or absence of CRP, SAP or PTX3;

FIG. 25 is a graph showing amounts of SC5b-9 in reaction supernatants formed by incubation of HEK293 cells expressing CL-P1 or pcDNA3.1 control vector together with aHUS patient serum or aHUS patient serum supplemented with CFH in the presence or absence of CRP, SAP or PTX3;

FIG. 26 shows fluorescent micrographs of HEK293 cells expressing CL-P1, LOX-1, SR-A1 or pcDNA3.1 control vector after incubation together with aHUS patient serum in the presence of CRP, in which CL-P1, LOX-1, SR-A1 and C5b-9 are fluorescently detected;

FIG. 27 is a graph showing C5b-9 deposition amounts formed by incubation of CL-P1 extracellular domain or BSA together with human complement serum or aHUS patient serum or aHUS patient serum to which CFH or sCR1 is added in the presence or absence of CRP, SAP or PTX3 in the ELISA system;

FIG. 28 is a graph showing C5b-9 deposition amounts formed by incubation of CL-P1 extracellular domain or BSA together with CFH depleted serum or CFH depleted serum to which sCR1 is added in the presence or absence of CRP, SAP or PTX3 in the ELISA system;

FIG. 29 is a graph showing C5b-9 deposition amounts formed by incubation of CL-P1 extracellular domain or BSA together with C4BP depleted serum or C4BP depleted serum to which sCR1 is added in the presence or absence of CRP, SAP or PTX3 in the ELISA system;

FIG. 30 is a graph showing C5b-9 deposition amounts formed by incubation of HEK293 cells expressing CL-P1 or pcDNA3.1 control vector together with aHUS patient serum or aHUS patient serum supplemented with sCR1 in the presence or absence of CRP;

FIG. 31 is a graph showing C5b-9 deposition amounts formed by incubation of HEK293 cells expressing CL-P1 or pcDNA3.1 control vector together with CFH depleted serum or CFH depleted serum supplemented with sCR1 in the presence or absence of CRP, SAP or PTX3; and

FIG. 32 is a graph showing C5b-9 deposition amounts formed by incubation of HEK293 cells expressing CL-P1 or pcDNA3.1 control vector together with C4BP depleted serum or C4BP depleted serum supplemented with sCR1 in the presence or absence of CRP, SAP or PTX3.

### DETAILED DESCRIPTION OF THE INVENTION

A first aspect of the present invention is a method for examining a complement system of a subject including:

a reaction step of letting a scavenger receptor or a functional equivalent thereof, a pentraxin family protein or a functional equivalent thereof and a blood sample collected from a subject coexist in vitro; and a detection step of detecting complement activation amplifying reaction and/or complement activation late-phase reaction induced by interaction between the scavenger receptor or the functional equivalent thereof and the pentraxin family protein or the functional equivalent thereof from a reactant after the reaction step.

The scavenger receptor is a transmembrane-type receptor protein that incorporates denatured LDL (low density lipoprotein) into cells, and is an important molecule involved in various physiological functions typified by formation of foamy macrophages in progress of an arteriosclerotic lesion. The term “scavenger receptor” used herein means a human scavenger receptor unless otherwise noted.

Six scavenger receptors including CL-P1 (Collectin placenta 1), SR-AI (scavenger receptor-AI), LOX-1 (lectin-like oxidized LDL receptor-1), SREC (scavenger receptor expressed by endothelial cells), CD36 and CD86 have been reported so far, and genes respectively encoding these have been cloned. In DDBJ/EMBL/GenBank which is a nucleotide sequence database, CL-P1 is registered as the accession No. AB005145, SR-AI as BC063878, LOX-1 as AB102861, SREC as BC039735, CD36 as M24795, and CD86 as KU284848.

In the present invention, any of these six scavenger receptors can be used. Besides these six scavenger receptors, functional equivalents thereof can be used in the present invention. Here, a functional equivalent of a scavenger receptor means a mutant, a modified body, and a peptide fragment having a function of inducing complement activation amplifying reaction and/or complement activation late-phase reaction by interaction with a pentraxin family protein as will be described later. Examples of the mutant include scavenger receptors having the amino acid sequence in which one or more amino acid residues are deleted, added, or substituted by genetic polymorphism or genetic engineering approach, examples of the modified body include scavenger receptors modified with a sugar chain, polyethylene glycol, a labeled compound or other chemical substance, and examples of the peptide fragment include receptor fragments containing an extracellular domain of the scavenger receptor. Hereinafter, a scavenger receptor or a functional equivalent thereof is indicated by SR unless otherwise noted.

SR that is preferably used in the examining method of the present invention is CL-P1, SR-AI or LOX-1, or a functional equivalent thereof, and CL-P1 or a functional equivalent thereof is more preferred.

SR can be separated or purified from natural cells expressing the same. SR for use can be prepared as a recombinant protein by using nucleotide sequence information and amino acid sequence information registered in public databases and a genetic engineering method known in the art.

The pentraxin family is a super family of cyclic multimeric protein formed of a plurality of associated subunits each having a PTX domain containing a characteristic motif of eight amino acid residues that is called pentraxin signature. The term “pentraxin family” herein means a human pentraxin family unless otherwise noted.

Examples of the pentraxin family protein include C-reactive protein (C reactive protein, CRP), and serum amyloid P component (Serum Amyloid P component, SAP) that are known as inflammatory proteins and pentraxin 3 (PTX3) expressed in endothelial cells. The genes respectively encoding CRP, SAP and PTX3 have been cloned, and the nucleotide sequences are registered as the accession numbers X56692, M10944, and X63053 respectively in DDBJ/EMBL/GenBank.

In the present invention, any of these three pentraxin family proteins can be utilized. Besides these three pentraxin family proteins, the functional equivalents of these can also be used in the present invention. Here, a functional equivalent of a pentraxin family protein means a mutant, a modified body, and a peptide fragment having a function of inducing complement activation amplifying reaction and/or complement activation late-phase reaction by interaction with SR as described above. Examples of the mutant include pentraxin family proteins having the amino acid sequence in which one or more amino acid residues are deleted, added, or substituted by genetic polymorphism or genetic engineering approach, examples of the modified body include pentraxin family proteins modified with a sugar chain, polyethylene glycol, a labeled compound or other chemical substance, and examples of the peptide fragment include fragments containing B-face which is a binding site to SR. Hereinafter, a pentraxin family protein or a functional equivalent thereof is indicated by PTXs unless otherwise noted.

PTXs for use can be separated or purified from blood. PTXs for use can be prepared as a recombinant protein by using nucleotide sequence information and amino acid sequence information registered in public databases and a genetic engineering method known in the art.

The examining method of the present invention includes a reaction step of letting SR, PTXs and a blood sample collected from a subject (hereinafter, referred to as blood sample) coexist. One preferred example of the reaction step is a reaction step of letting SR existing on mammalian cells, PTXs and a blood sample coexist. Another example of the reaction is a reaction step of letting SR immobilized on a laboratory dish, a well plate or other appropriate support, particularly, a receptor fragment that contains an extracellular domain of a scavenger receptor and is immobilized on a support, PTXs and a blood sample coexist.

The mammalian cells used in the present invention are preferably human cells. By using human cells, it is possible to avoid nonspecific activation of the complement system (increase in background) that is independent of the existence of SR on the cells. Examples of the human cells include human embryonic kidney-derived cells, human cardiac muscle cells, human endothelial cells, human epithelial cells, human fibroblasts, and human smooth muscle cells. Particularly preferred human cells are human endothelial cells or human embryonic kidney-derived cells.

Mammalian cells for use can be adhered to a laboratory dish, a well plate or other appropriate support that allows adhesion of cells.

SR existing on mammalian cells may be SR that are expressed on the surface of naturally existing cells, but are preferably SR expressed on the surface of mammalian cells, in particular, human cells transformed by an expression vector into which a gene encoding SR is incorporated. Examples of such SR include CL-P1 expressed in HEK293 cells that are prepared by transforming human cells with a vector expressing the full length of CL-P1 constructed according to the method described in Jang et al. (J. Biol. Chem., 2009, 284, 3956-3965). SR other than CL-P1 can be prepared in a manner similar to the method described in the above Jang et al. except for replacing the gene encoding CL-P1 by a gene encoding other SR.

SR immobilized on a support can be prepared by placing a solution containing SR, preferably a solution containing a receptor fragment containing an extracellular domain of a scavenger receptor on a laboratory dish, a well plate or other appropriate support capable of adsorbing protein, and incubating the support in appropriate conditions.

The blood sample may be blood as is collected from a subject, or may be serum or plasma separated from blood. For the purpose of examining abnormality of a specific complement protein, a blood sample from/to which complement protein is depleted or supplemented may be used. The complement protein depleted blood sample can be prepared, for example, by subjecting blood, serum or plasma collected from a subject to an affinity chromatography to which a specific antibody against the complement protein is bound.

The reaction of letting SR, PTXs and a blood sample coexist is conducted in an appropriate medium, typically in a buffer or a liquid medium used for cell culture, preferably in a buffer or a liquid medium in physiologic conditions where protein does not lose its activity. When SR existing on mammalian cells is used, the medium is preferably a buffer or a liquid medium in physiologic conditions where cells can be alive. When SR immobilized on a support is used, the medium is preferably a buffer in physiologic conditions where protein does not denature.

The examining method of the present invention includes a detection step of detecting complement activation amplifying reaction and/or complement activation late-phase reaction induced by interaction between SR and PTXs from the reactant after the reaction step.

The complement activation amplifying reaction can be detected by immunoassay targeting an amplifying reaction activation factor properdin, D factor (Complement Factor D, CFD), B factor (Complement Factor B, CFB), Ba (degradation product of CFB) and/or C3bBb (a conjugate of C3b which is a C3 degradation product and Bb which is a CFB degradation product), in which assay an antibody against each factor can be utilized. The complement activation late-phase reaction can be detected by immunoassay targeting C5a, C5b and/or C5b-9, in which assay an antibody against each of them can be utilized. Preferably, detection of the complement activation amplifying reaction is conducted by an immunoassay targeting properdin or CFB, and detection of the complement activation late-phase reaction is conducted by an immunoassay targeting C5b-9. The immunoassay can be conducted by a common method known by a person skilled in the art, or by using a commercially available kit.

In the examining method of the present invention, it is preferred to detect complement activation early-phase reaction in addition to detecting the complement activation amplifying reaction and/or the complement activation late-phase reaction. Preferably, detection of the complement activation early-phase reaction is conducted by targeting C3 activation, namely C3b or C3d which is a C3 degradation product is targeted for measurement.

Localization of complement protein at the time of activation differs depending on the individual complement protein, and for example, properdin, CFD, CFB, C3bBb, C3b, and C3d exist on SR or cellular membrane, Ba, and C5a exist in a liquid phase, and C5b-9 exists as TCC on SR or cellular membrane, or exists as SC5b-9 in a liquid phase. Therefore, the activated complement protein can be quantified by subjecting a support or cells after completion of the reaction step to measurement in the case that the complement protein existing on SR or cellular membrane when activated is a measurement target, or by subjecting a liquid phase after completion of the reaction step to measurement in the case that the complement protein existing in a liquid phase when activated is a measurement target. Further, according to the examining method of the present invention using SR that exists on mammalian cells in the reaction step, it is possible to obtain information of co-localization of each complement protein in cells.

The examining method of the present invention is capable of examining whether or not the complement system contained in the blood sample is activated, whether or not there is abnormality in the complement system, and which complement protein is involved in the abnormality. Concretely, various information can be obtained by executing the examining method of the present invention using a blood sample in combination with the examining method of the present invention using the blood sample having undergone a specific pretreatment and/or the examining method of the present invention including the reaction step in which a complement protein that is suspected of abnormality is added. Hereinafter, practical examples and obtainable information of the examining method of the present invention are shown.

### EXAMINATION EXAMPLE 1

As a result of conducting the examining method of the present invention, if

properdin or CFB is detected,

C5b-9 is detected, and

C3b or C3d is detected;

it is determined that the blood sample possibly has deficiency or functional depression of a complement regulatory factor (CFH, C4BP or CFI) of the complement activation late-phase reaction, or an abnormal enhancement of an activation factor (C3, CFB) of the amplifying reaction. In this case, by additionally conducting the method of the present invention including the reaction step in which a complement regulatory factor is added, or the reaction step in which a neutralizing antibody against an activation factor of the amplifying reaction is added, it is possible to identify the cause of the abnormality.

### EXAMINATION EXAMPLE 2

As a result of conducting the examining method of the present invention, if

properdin or CFB is detected,

C5b-9 is not detected, and

C3b or C3d is detected; and

as a result of additionally conducting the method of the present invention using a blood sample from which a complement regulatory factor is artificially removed, if

C5b-9 is detected;

it is determined that any complement system protein contained in the blood sample is normal, and is regulated normally.

### EXAMINATION EXAMPLE 3

As a result of conducting the examining method of the present invention, if

properdin or CFB is detected,

C5b-9 is not detected, and

C3b or C3d is not detected, or detected slightly; and

as a result of additionally conducting the method of the present invention including the reaction step in which a complement protein (C1q, C2, C4) involved in the complement activation early-phase reaction is added, if

C3b or C3d is detected, or the detected amount increases;

it is determined that the blood sample possibly has deficiency or functional depression regarding the added complement protein involved in the complement activation early-phase reaction.

### EXAMINATION EXAMPLE 4

As a result of conducting the examining method of the present invention, if

properdin or CFB is not detected,

C5b-9 is not detected, and

C3b or C3d is not detected, or detected slightly; and

as a result of additionally conducting the method of the present invention including the reaction step in which an activation factor of the complement activation amplifying reaction is added, if

C3b or C3d is detected, or the detected amount increases;

it is determined that the blood sample possibly has deficiency or functional depression regarding the added activation factor of the complement activation amplifying reaction.

### EXAMINATION EXAMPLE 5

As a result of conducting the examining method of the present invention, if

properdin or CFB is not detected,

C5b-9 is not detected,

C3b or C3d is not detected, or detected slightly; and as a result of additionally conducting the method of the present invention including the reaction step in which an activation factor of the complement activation amplifying reaction is added, if

C3b or C3d is detected, and

C5b-9 is not detected, and

as a result of additionally conducting the method of the present invention using a blood sample from which a complement regulatory factor is artificially removed, if

C5b-9 is detected,

it is determined that the blood sample is normal in the complement activation late-phase reaction, but possibly has deficiency or functional depression regarding the added activation factor of the complement activation amplifying reaction.

The above-described examination example 1 corresponds to the conventional collective measuring method, and a subject in which a complement is activated or abnormally enhanced can be easily discriminated by this practical example 1. Examination example 2 discriminates whether the complement system is normal, or whether there is some abnormality that prevents activation of the complement system, examination examples 3 and 4 discriminate whether there is abnormality in a specific complement protein, and examination example 5 discriminates which one of complement proteins has abnormality.

A second aspect of the present invention relates to a kit for examining a complement system, containing SR and PTXs. In particular, a kit containing SR expressing cells and PTXs is preferred.

SR and PTXs contained in the kit of the present invention are as described in the foregoing first aspect. The kit may contain a substance that can be used for detecting complement protein, for example, a specific antibody against complement protein, a secondary antibody against the specific antibody, a detection reagent, a coloring reagent, a buffer and other any reagents.

A third aspect of the present invention relates to a method for testing a test substance for the ability to activate or the ability to suppress a complement system including: a test substance reaction step of letting SR, PTXs, a blood sample, and a test substance coexist in vitro; a control reaction step of letting SR, PTXs, and a blood sample coexist in vitro; a detection step of detecting complement activation amplifying reaction and/or complement activation late-phase reaction induced by interaction between SR and PTXs from a reactant after each reaction step; and a determination step of determining that the test substance has the ability to activate the complement system when the complement activation amplifying reaction and/or the complement activation late-phase reaction detected from the reactant after the test substance reaction step is larger than the complement activation amplifying reaction and/or the complement activation late-phase reaction detected from the reactant after the control reaction step, and determining that the test substance has the ability to suppress the complement system when the complement activation amplifying reaction and/or the complement activation late-phase reaction detected from the reactant after the test substance reaction step is smaller than the complement activation amplifying reaction and/or the complement activation late-phase reaction detected from the reactant after the control reaction step.

Both of the test substance reaction step and the control reaction step, and the detection step in the testing method of the present invention are as described in the foregoing first aspect except that the test substance is caused to coexist in the test substance reaction step.

The blood sample used in the test substance reaction step and the control reaction step may be a blood sample derived from a healthy subject, or a blood sample derived from a patient. However, since blood of a healthy subject contains complement regulatory factors, the complement activation late-phase reaction may not be detected or detected at a low level, and thus the test substance having low activity sometimes cannot be tested with satisfactory sensitivity. Therefore, particularly in the case of testing the ability to activate or the ability to suppress the complement activation late-phase reaction, it is preferred to use a blood sample in which complement regulatory factors are depleted or reduced, namely a blood sample derived from a health person in which complement regulatory factors are depleted or reduced, or a blood sample derived from a patient for which lack or reduction in complement regulatory factors is known. In the case of testing the ability to activate or the ability to suppress specific complement activation reaction, the test may be conducted by using a blood sample in which the complement protein involved in the reaction is depleted or enriched.

In the determination step, the complement activation amplifying reaction and/or the complement activation late-phase reaction detected from a reactant after the test substance reaction step, and the complement activation amplifying reaction and/or the complement activation late-phase reaction detected from a reactant after the control reaction step are compared with each other. When the former is larger than the latter, namely, when the reactant after the test substance reaction step contains a larger amount of complement protein which is an index for the complement activation than the reactant after the control reaction step, it can be determined that the test substance has the ability to activate the complement system. On the other hand, when the former is smaller than the latter, namely, when the reactant after the control reaction step contains a larger amount of complement protein which is an index for the complement activation than the reactant after the test substance reaction step, it can be determined that the test substance has the ability to suppress the complement system. The test substance determined to have the ability to activate or the ability to suppress the complement system is a potential pharmaceutical for a patient having abnormality in the regulatory mechanism of the complement system.

In the testing method of the third aspect, the ability to activate or the ability to suppress the complement system can also be tested by further detecting complement activation early-phase reaction, and comparing the magnitude of the complement activation early-phase reaction between the reactant of the test substance reaction step and the reactant of the control reaction step.

The present invention will be described more specifically by the following examples, however, it is to be noted that the present invention is not limited to these examples.

### EXAMPLES

### Materials and Methods

### Cells

CHO/ldlA7 cells lacking functional LDL receptors were distributed from Dr. M. Krieger (MIT). Human embryonic kidney (HEK293) cells were purchased from ATCC.

### Proteins and Reagents

Used reagents and the respective suppliers are as follows: native CRP, C1q depleted serum and anti-rabbit IgG HRP (Merck Millipore); recombinant human pentraxin 2 (SAP), recombinant human pentraxin 3 (PTX3), soluble complement receptor 1 (sCR1, recombinant human CD35), biotinylated anti-mouse pentraxin 2 (cross-reacting with human pentraxin 2), biotinylated anti-human PTX3 (R&D Systems); purified native C1q, purified human CFH, purified human properdin, purified human CFB, purified human CFI, rabbit anti-human C5b-9 antibody, goat anti-human CFH antibody, goat anti-human CFB antibody, goat anti-human properdin antibody, properdin depleted serum, CFB depleted serum, CFI depleted serum and CFH depleted serum (Complement Technology); mouse anti-human C4BP antibody, purified human C4BP and MicroVue SC5b-9 plus EIA kit (Quidel); HAM's F-12, Dulbecco's minimum essential medium (DMEM)-high glucose, fetal bovine serum (FBS) and human complement serum (Sigma-Aldrich); anti-myc monoclonal antibody, Alexa Fluor 555 antibody labeling kit, EZ-Link Sulfo-NHS-LC-LC-biotin and Alexa Fluor conjugate antibody (Invitrogen); rabbit anti-human C3d antibody (Dako).

C4BP depleted serum was prepared by letting 40% human complement serum diluted in PBS pass through a HisTrap NHS activation column (1 mL; GE Healthcare) to which about 10 mg of ovine anti-C4BP IgG antibody (Abcam) is bound. Absence of C4BP in the obtained plasma was confirmed by Western blot analysis. Serum of atypical hemolytic uremic syndrome (aHUS) patient was provided by Professor Yoshihiko Hidaka (SHINSYU UNIVERSITY), and as a result of nucleotide sequence analysis, heterozygous G3717A mutation, and heterozygous polymorphism of C-257T, A2089G, G2881T and G1492A were observed in FH1. Anti-CFH antibody was not detected in this patient serum.

### Preparation of Scavenger Receptor Expressing Cells

According to the method described in Document (S. Jang et al., J. Biol. Chem., 2009, 284, 3956-3965), cDNA encoding human full-length CL-P1 was cloned into pcDNA3.1/myc-HisA expression vector. Human LOX-1 and SR-A1 were also cloned into pcDNA3.1/myc-HisA expression vector in the same manner. CHO/ldlA7 cells were cultured in HAM's F-12 medium containing 5% heat-inactivated FBS, and HEK293 cells were cultured in DMEM-high glucose medium containing 10% FBS at 37° C., 5% CO₂. CHO/ldlA7 cells were plated on poly-L-lysine-coated glass dishes (Iwaki, Japan), and HEK293 cells were plated on collagen-coated glass dishes (Iwaki, Japan), and after 24 hours, the vectors were introduced into respective cells by using Lipofectamine LTX transfection reagent (Invitrogen). After 6 hours from transfection, the medium was replaced, and after 24 hours, the cells were used in the following assay.

### Binding Assay Between CL-P1 and PTXs Using ELISA System

cDNA encoding extracellular domain of human CL-P1 (59-742 in CL-P1 amino acid sequence) having insulin leader peptide followed by FLAG tag added to the N terminus was subcloned into pcDNA3.1 vector. The plasmid was transfected into Expi 293-F cells (Invitrogen) using ExpiFectamine 293 transfection reagent. After 7 days of cell culture, the culture supernatant was collected, and a soluble recombinant human CL-P1 extracellular domain was purified by using anti-FLAG M2 affinity gel (Sigma).

CL-P1 extracellular domain (0.1 μg) or BSA (0.1 μg) (Thermo Scientific) was immobilized to a 96-well immunoplate (Maxisorp, Thermo Fisher Scientific) by incubating overnight at 4° C. in a coating buffer (15 mM Na₂CO₃, 35 mM NaHCO₃, 0.05% NaN₃, pH 9.6). After washing three times with TBSTC (10 mM Tris-HCl, 150 mM NaCl, 0.05% Tween 20, 5 mM CaCl₂, pH 7.4), the plate was blocked with BlockAce/PBS (DS Pharma Biomedical) at 37° C. for 1 hour. After washing with TBSTC, CRP, SAP or PTX3 or thermally denatured CRP, SAP or PTX3 obtained by boiling for 5 minutes was added to each well, and the plate was incubated at 37° C. for 1 hour. Then biotin conjugated antibody was added and the plate was left at 37° C. for 1 hour, and Elite ABC kit (Vector laboratories) was added and the plate was incubated for another 1 hour. After washing, 100 μL of SureBlue TMB micro well peroxidase substrate (Kirkegaad & Perry Laboratories) was applied to each well, and the plate was incubated at room temperature for 15 minutes. Finally, 100 μL of 1 M phosphoric acid was added to stop the reaction and the absorbance at 450 nm was read with a model 680 microplate reader (Bio-Rad Lab.).

### Binding Assay Between CL-P1 Expressed on CHO/ldlA7 Cell Surface, and PTXs

CL-P1 expressing CHO/ldlA7 cells were incubated in an ice-cooled HAM's F12 medium/10 mM HEPES containing 10 μg/mL Alexa 555-CRP, SAP or PTX3 at 4° C. for 1 hour. Cells were fixed at room temperature for 30 minutes using 4% phosphate buffered formalin (Wako Pure Chemical industries), and then washed, and incubated together with anti-myc antibody, or a combination of anti-myc antibody and biotinylated anti-mouse pentraxin 2 antibody (cross-reacting with human pentraxin 2) or biotinylated anti-PTX3 antibody at room temperature for 30 minutes. After washing, cells were incubated together with Alexa 488-anti mouse IgG antibody, or a combination of Alexa 488-anti mouse IgG antibody and streptavidin Alexa 555 conjugate at room temperature for 30 minutes. Nuclear counterstaining was performed with Hoechst 33342 (Invitrogen). The images were taken using a fluorescent microscope (BZ-9000, Keyence) at 40-fold magnification. Signal intensity of each image was calculated by using the BZ-HIC program (Keyence).

### Complement Activation Assay Using ELISA System

To each well of a 96-well immunoplate, 100 μL of CL-P1 extracellular domain (5 μg/mL) or heat inactivated BSA (5 μg/mL) diluted in a coating buffer was added, and immobilized by overnight incubation at 4° C. After washing three times with PBS, the plate was blocked with 2% BSA in PBS at 37° C. for 1 hour. After washing, to each well, Veronal buffer (0.82 mM MgCl₂, 145.45 mM NaCl and 0.25 mM CaCl₂, 3.11 mM barbitol, 1.8 mM sodium barbitol) containing 0.1% gelatin with addition or without addition of 20 μg/mL (full serum) of CRP, SAP or PTX3 was added together with 1% human complement serum or various complement depleted serum or aHUS patient serum, and the plate was incubated at 37° C. for 1 hour. Then to each well after washing, 3.5 μg/mL of rabbit anti-human C3d antibody or rabbit polyclonal anti-human C5b-9 antibody diluted in PBS containing 1% BSA, 0.05% Tween 20 was added, and the plate was incubated, and then horseradish peroxidase (HRP)-conjugated anti-rabbit IgG antibody (1:5000) was added and the plate was incubated. Then peroxidase activity was measured in the same manner as described above by letting the plate react with SureBlue TMB microwell peroxidase substrate, and thus the amounts of C3d and C5b-9 generated by complement activation were measured.

In part of assays, C1q (200 μg/mL full serum), properdin (6 μg/mL full serum), CFB (200 μg/mL full serum), CFH (400 μg/mL full serum), C4BP (160 μg/mL full serum) or CFI (35 μg/mL full serum) was added to the corresponding depleted serum. In Example 6, an inhibitory assay in which sCR1 (10.65 μg/mL) is further added was conducted.

### Complement Activation Assay Using HEK293 Cell System

CL-P1 expressing HEK293 cells were incubated in a DMEM-high glucose medium containing 10% human complement serum or various complement depleted serum or aHUS patient serum in the presence or absence of 20 μg/mL (full serum) of CRP, SAP or PTX3 at 37° C. for 1 hour. The cells were fixed with 4% phosphate-buffered paraformaldehyde, and immunostained with rabbit anti-human C3d antibody (3.5 μg/mL) or rabbit polyclonal anti-human C5b-9 antibody in combination with Alexa Fluor 594 anti-rabbit IgG antibody, and C3d and C5b-9 generated by complement activation were detected. The cells were imaged by using a fluorescent microscope, and signal intensities were calculated by a BZ-HIC program, and a mean fluorescent intensity (MFI) which is a mean value of the signal intensities was determined as a quantitative value of each complement protein.

Likewise the ELISA system, in part of assays, C1q (200 μg/mL full serum), properdin (6 μg/mL full serum), CFB (200 μg/mL full serum), CFH (400 μg/mL full serum), C4BP (160 μg/mL full serum) or CFI (35 μg/mL full serum) was added to the corresponding depleted serum. In Example 6, an inhibitory assay in which sCR1 (10.65 μg/mL) is further added was conducted.

After fixing the cells incubated with 10% human complement serum in the presence or absence of 20 μg/mL (full serum) of CRP, SAP or PTX3, the cells were incubated with goat polyclonal anti-human CFH antibody, goat anti-human properdin antibody, goat anti-human CFB antibody or anti-human C4BP antibody, followed by Alexa-conjugated secondary antibody at room temperature for 30 minutes. By imaging the cells and calculating the signal intensity, recruitment of CFH, C4BP, CFB and properdin was evaluated.

Further, a medium of cells incubated with 10% CFH depleted serum or 5% aHUS patient serum or these serums supplemented with purified CFH (400 μg/mL full serum), in the presence or absence of 20 μg/mL (full serum) of CRP was collected, and SC5b-9 generated by complement activation was quantified by using a MicroVue SC5b-9 plus EIA kit.

### Statistical Analysis

Statistical analysis was conducted using the unpaired two-tailed Student's t test included in the JMP statistics software package (version 7, SAS). In Examples, data are mean±standard error. In graphs, the asterisk indicates a significant difference (p<0.0001), and ns indicates that there is no significant difference.

### Example 1 Evaluation of Binding Between CL-P1 and PTXs

### 1-1. Evaluation Using ELISA System

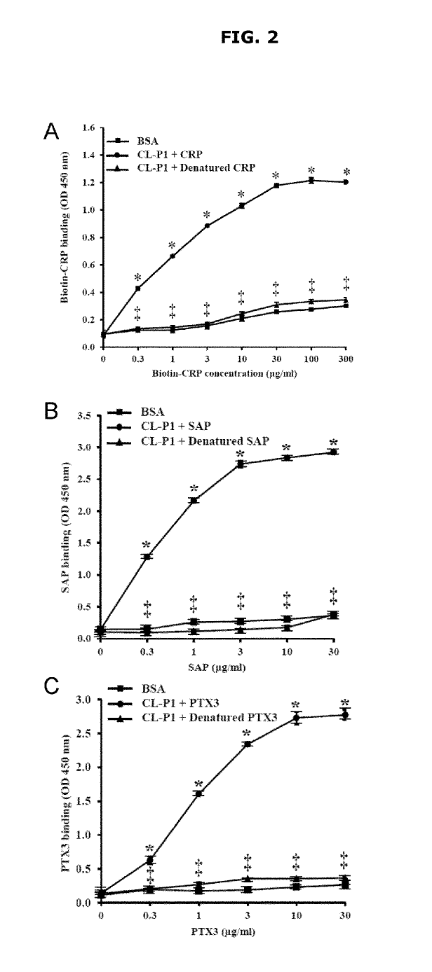

Binding between CL-P1 extracellular domain and PTXs (CRP, SAP and PTX3) was evaluated by using ELISA. The results are shown in FIGS. 2A to 2C. CRP, SAP and PTX3 bind to CL-P1 extracellular domain specifically and dose-dependently, and they lost the ability to bind to CL-P1 extracellular domain when they were heat-denatured.

### 1-2. Evaluation Using Cell System

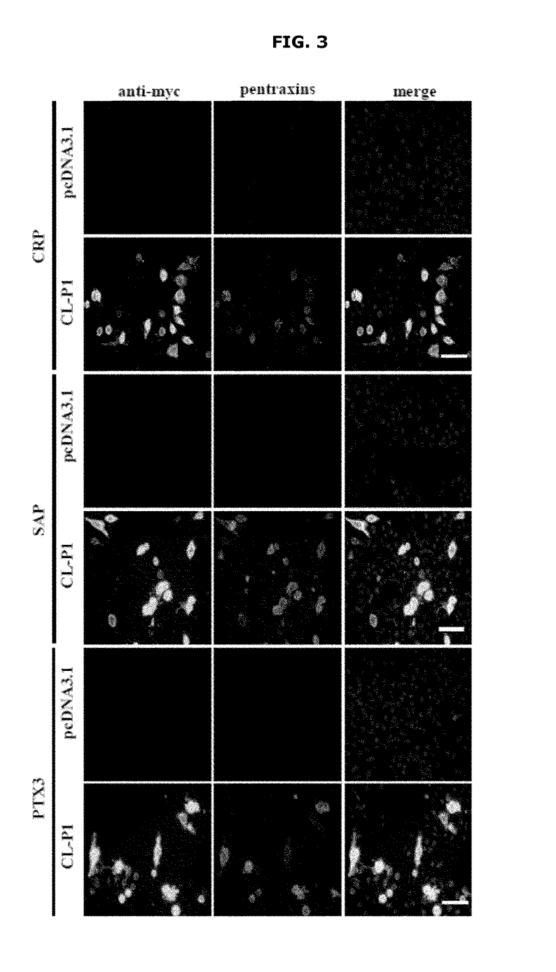

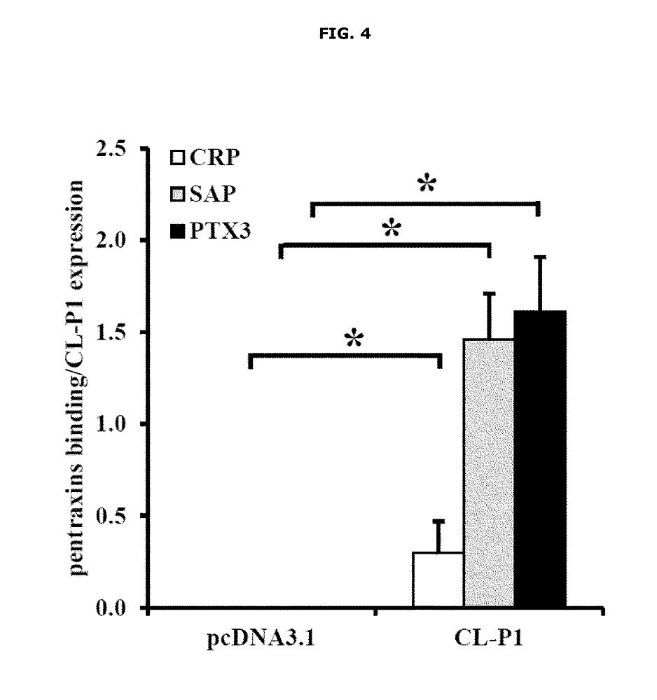

Binding between CL-P1 and PTXs was evaluated by using CHO/ldlA7 cells. As shown in FIG. 3, while CRP, SAP and PTX3 were co-localized with CL-P1 in CL-P1 expression cells, none of CRP, SAP, and PTX3 was detected in the cells expressing pcDNA3.1 control vector. This indicates that CRP, SAP and PTX3 bind to CL-P1 on the cell surface. A mean fluorescent intensity was calculated from each fluorescent image, and a binding amount of CRP, SAP or PTX3 relative to the CL-P1 expression amount was compared. The binding amount of CRP to CL-P1 was in a lower level compared with the binding amounts of SAP and PTX3 (FIG. 4).

### Example 2 Evaluation of Complement Activation Early-Phase Reaction Induced by Binding Between CL-P1 and PTXs

### 2-1. Evaluation of Complement Activation Early-Phase Reaction Using ELISA System

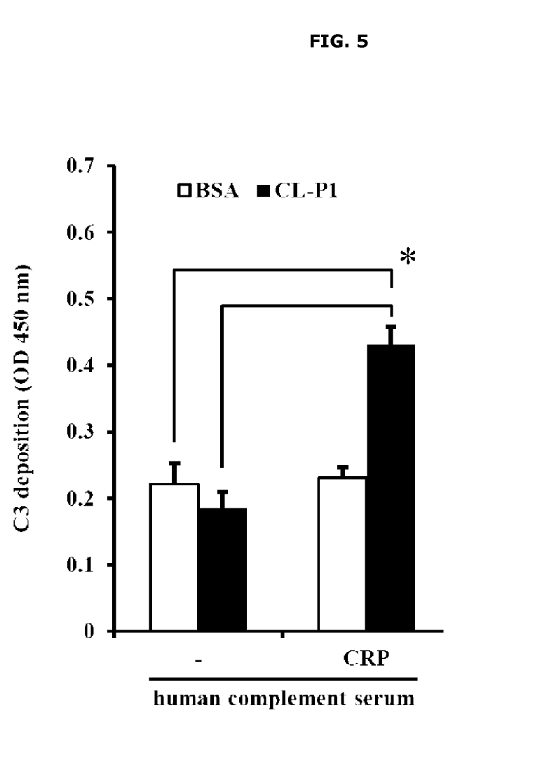

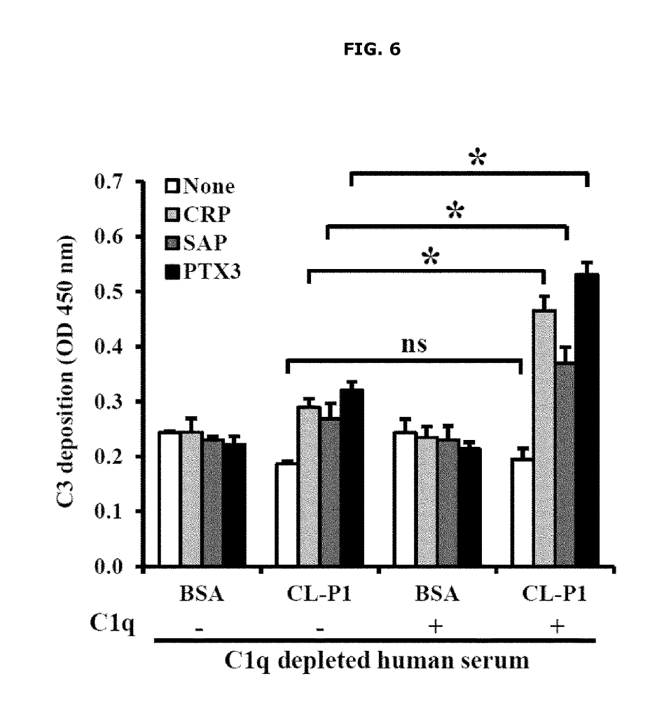

Induction of complement activation early-phase reaction by binding between CL-P1 extracellular domain and PTXs was evaluated by using ELISA. As shown in FIG. 5, in the presence of human complement serum, increase in C3d deposition amount depending on addition of CRP was observed in a CL-P1 extracellular domain-immobilized well. When C1q depleted serum was used, C3d deposition amount in a CL-P1 extracellular domain-immobilized well increased by addition of CRP, SAP or PTX3, and this increase in C3d deposition amount was further enhanced by using C1q depleted serum supplemented with C1q (FIG. 6). These indicate that CRP, SAP and PTX3 bind to extracellular domain of CL-P1, and activate the complement activation early-phase reaction via C1q to induce deposition of C3d.

### 2-2. Evaluation of Complement Activation Early-Phase Reaction Using Cell System

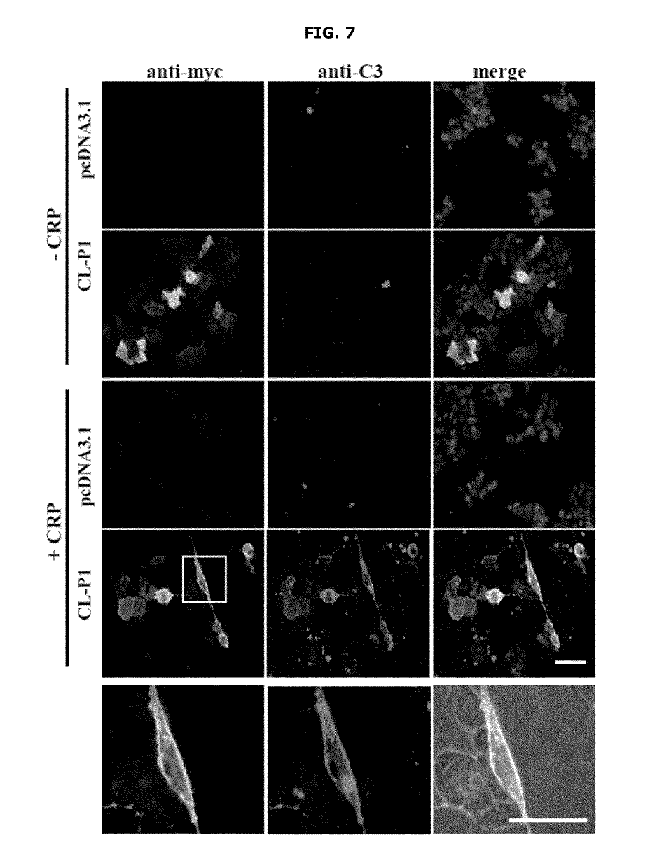

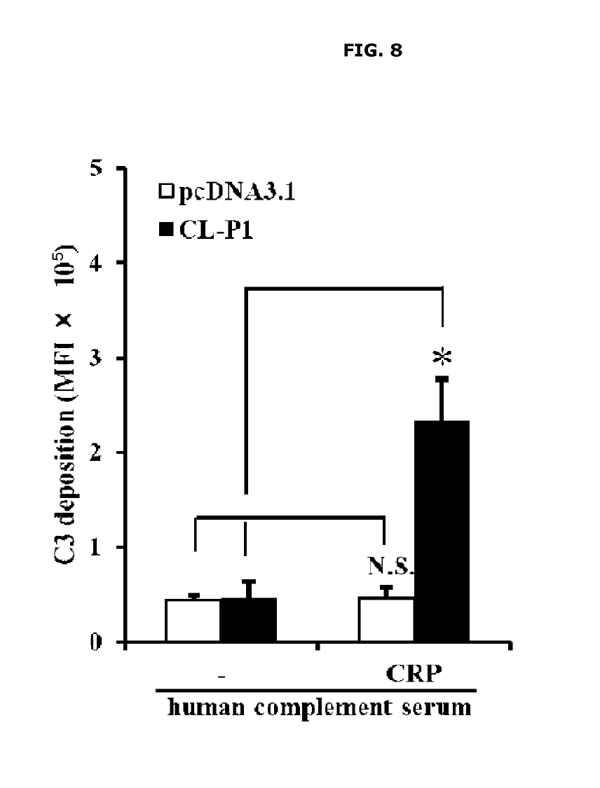

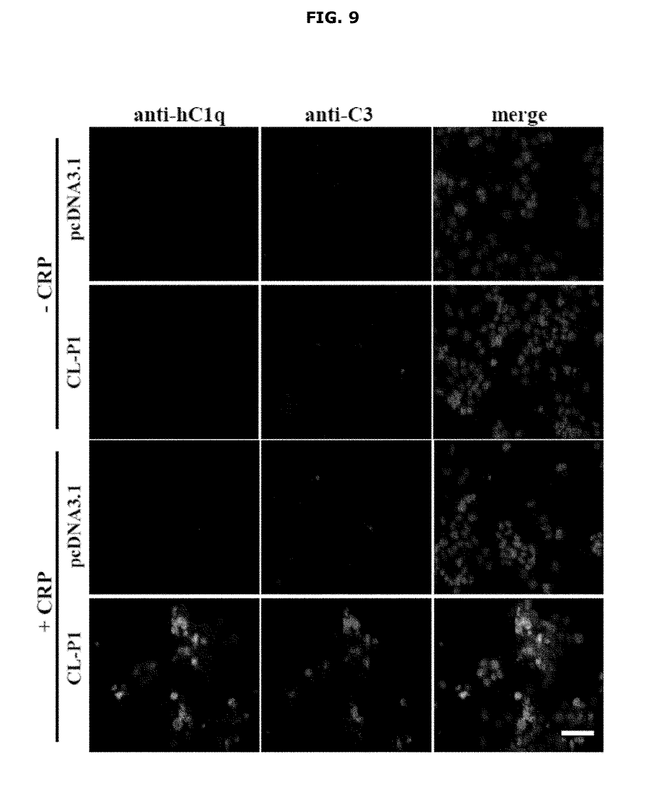

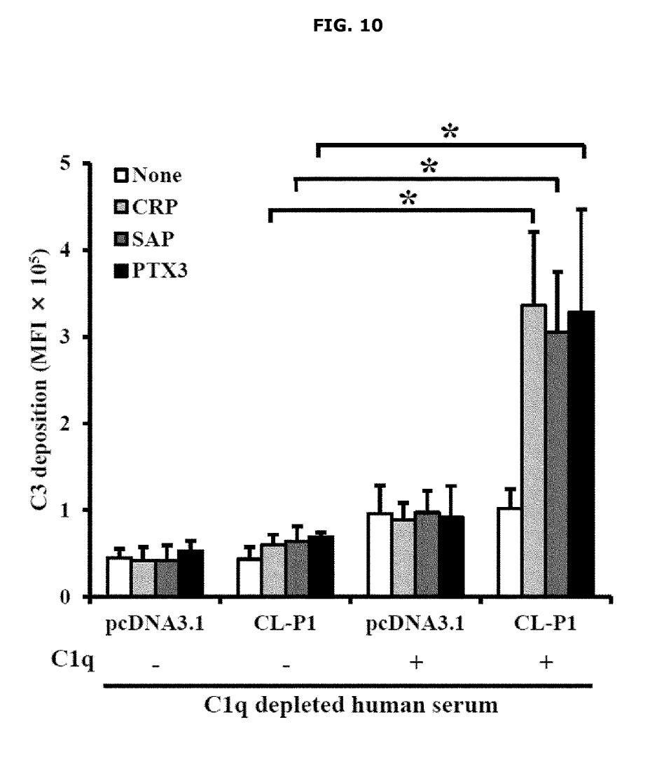

Induction of complement activation early-phase reaction by binding between CL-P1 and PTXs was evaluated by using CL-P1 expressing HEK293 cells. When human complement serum was used, increase in C3d deposition amount depending on addition of CRP was observed in CL-P1 expressing HEK293 cells (FIG. 7 to FIG. 9). At this time, C3d was co-localized with CL-P1 (FIG. 7) and C1q (FIG. 9), and existence of both of these was observed all over the cell surface. When C1q depleted serum not containing C1q was used, C3d deposition by addition of CRP, SAP and PTX3 did not occur, and C3d deposition recovered by supplementing depleted serum with C1q (FIG. 10). These indicate that CRP, SAP and PTX3 bind to CL-P1 on the surface of HEK293 cells, and activate the complement activation early-phase reaction via C1q to induce deposition of C3d on the cell surface.

### Example 3 Evaluation of Complement Activation Amplifying Reaction Induced by Binding Between CL-P1 and PTXs

### 3-1. Evaluation of Complement Activation Amplifying Reaction Via CFB Using ELISA System

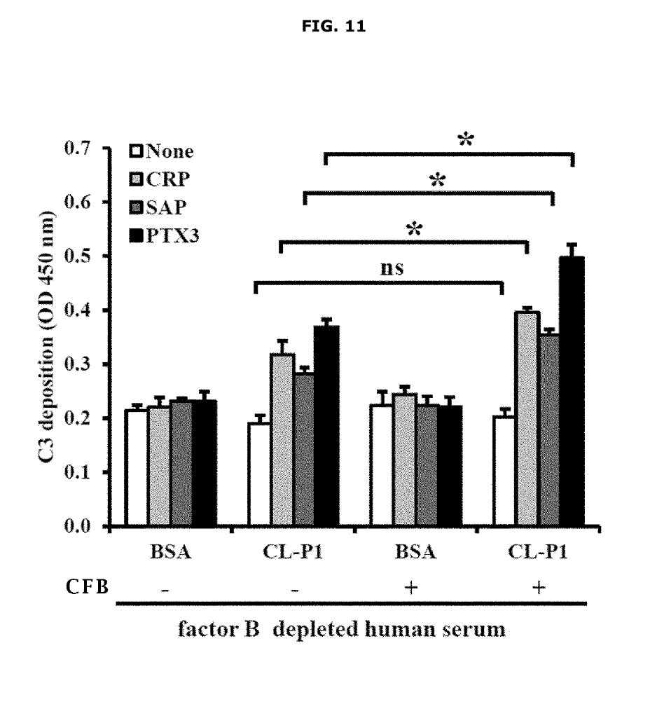

Induction of the complement activation amplifying reaction by binding between CL-P1 extracellular domain and PTXs was evaluated by using ELISA. When CFB depleted serum supplemented with CFB was used, further increase in C3d deposition amount depending on the addition of CRP, SAP and PTX3 in a CL-P1 extracellular domain-immobilized well was observed, as compared with the case where CFB depleted serum was used (FIG. 11). These indicate that CRP, SAP and PTX3 bind to extracellular domain of CL-P1, and activate the complement activation amplifying reaction via CFB to induce deposition of C3d.

### 3-2. Evaluation of Complement Activation Amplifying Reaction Via CFB Using Cell System

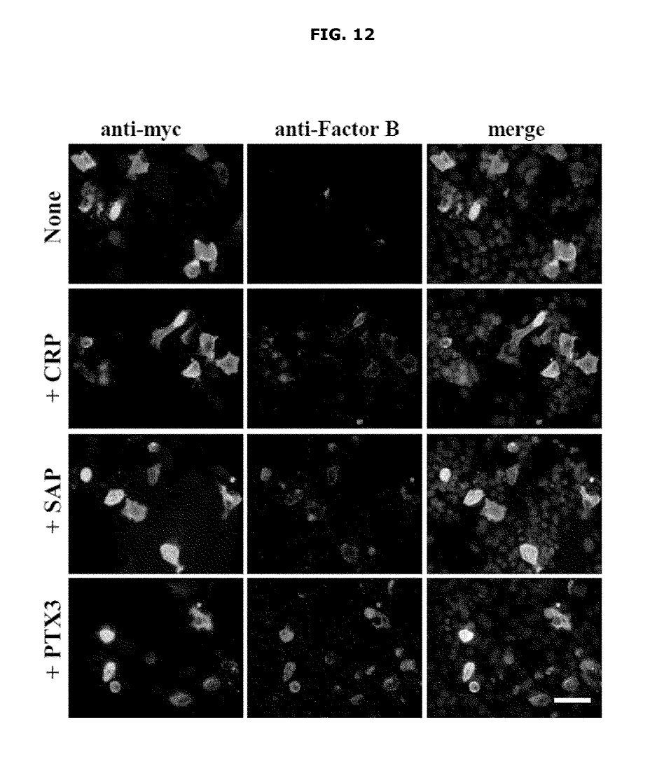

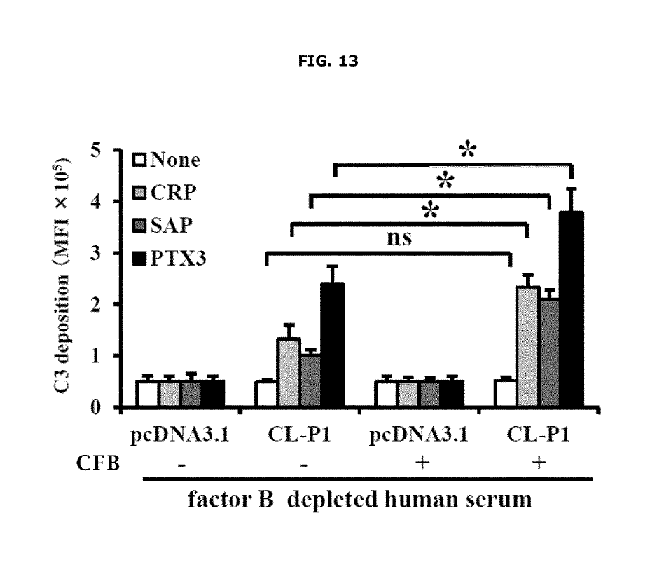

Induction of complement activation amplifying reaction by binding between CL-P1 and PTXs was evaluated by using CL-P1 expressing HEK293 cells. When human complement serum was used, recruitment of CFB depending on the addition of CRP, SAP or PTX3 was observed in CL-P1 expressing HEK293 cells (FIG. 12). When CFB depleted serum supplemented with CFB was used, further increase in C3d deposition amount depending on the addition of CRP, SAP or PTX3 was observed in CL-P1 expressing HEK293 cells, as compared with the case where CFB depleted serum was used (FIG. 13). These indicate that CRP, SAP and PTX3 bind to CL-P1 on the surface of HEK293 cells, and activate the complement activation amplifying reaction via CFB to induce deposition of C3d on the cell surface.

### 3-3. Evaluation of Complement Activation Amplifying Reaction Via Properdin Using ELISA System

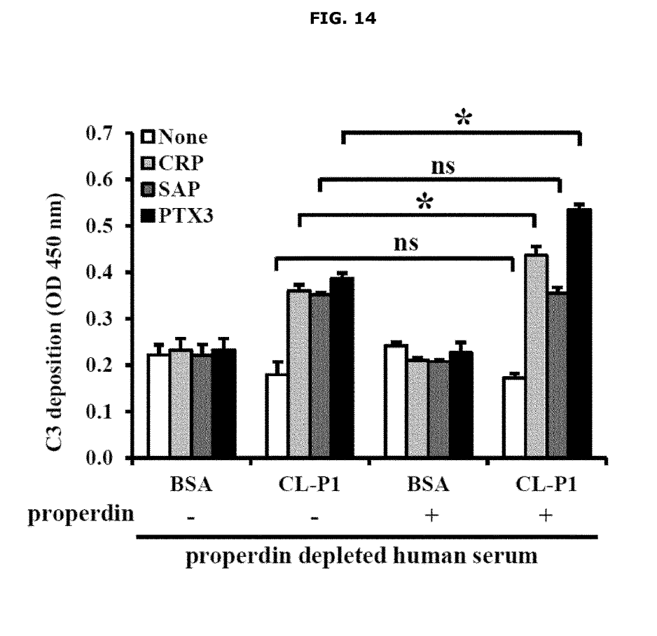

Induction of complement activation amplifying reaction by binding between CL-P1 extracellular domain and PTXs was evaluated by using ELISA. When properdin depleted serum supplemented with properdin was used, the C3d deposition amount in a CL-P1 extracellular domain-immobilized well further increased by addition of CRP or PTX3, and SAP addition did not influence on the C3d deposition amount, as compared with the case where properdin depleted serum was used (FIG. 14). These indicate that CRP and PTX3 bind to CL-P1 extracellular domain, and activate the complement activation amplifying reaction via properdin to induce deposition of C3d.

### 3-4. Evaluation of Complement Activation Amplifying Reaction Via Properdin Using Cell System

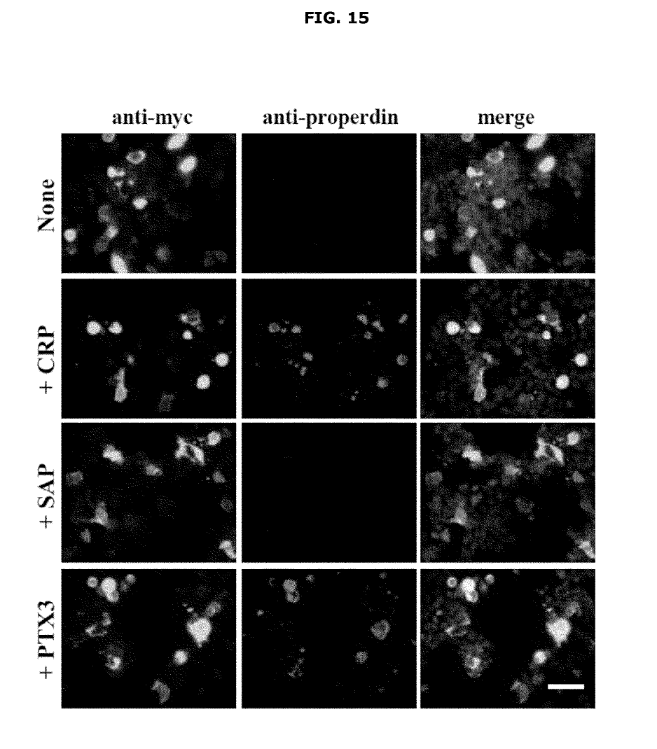

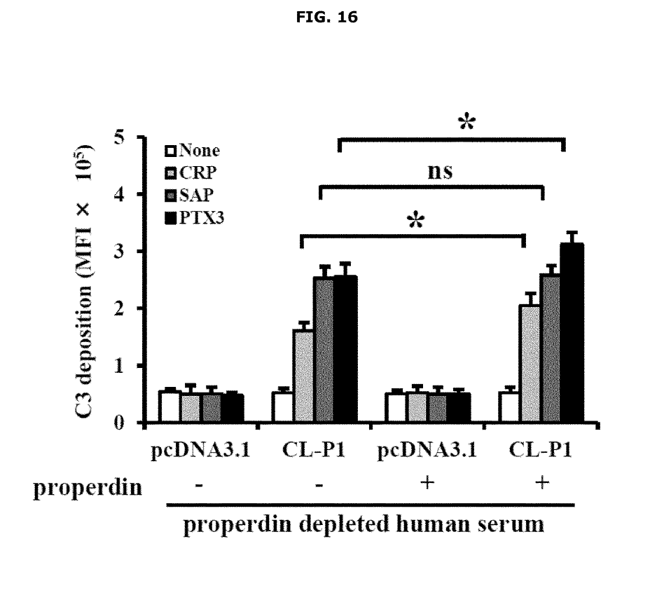

Induction of complement activation amplifying reaction by binding between CL-P1 and PTXs was evaluated by using CL-P1 expressing HEK293 cells. When human complement serum was used, recruitment of properdin depending on addition of CRP or PTX3 was observed in CL-P1 expressing HEK293 cells, and this phenomenon was not observed when SAP was added (FIG. 15). When properdin depleted serum supplemented with properdin was used, the C3d deposition amount in CL-P1 expressing HEK293 cells further increased by addition of CRP or PTX3, but addition of SAP did not influence on the C3d deposition amount, as compared with the case where properdin depleted serum was used (FIG. 16). These indicate that CRP and PTX3 bind to CL-P1 on the surface of HEK293 cells, and activate the complement activation amplifying reaction via properdin to induce deposition of C3d on the cell surface.

### Example 4 Evaluation of Complement Activation Late-Phase Reaction Induced by Binding Between CL-P1 and PTXs

### 4-1. Recruitment of Complement Regulatory Factor

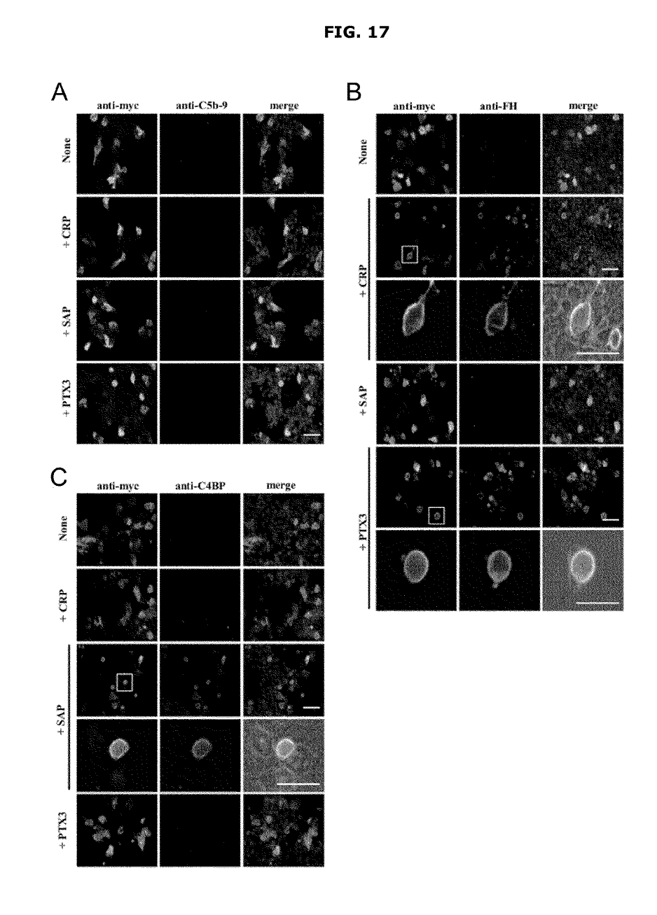

In CL-P1 expressing HEK293 cell system, C5b-9 deposition was not observed both in the presence and in the absence of CRP, SAP or PTX3 when human complement serum was used (FIG. 17A). Since CFH which is a complement regulatory factor was co-localized with CL-P1 in the presence of CRP or PTX3, and C4BP was co-localized with CL-P1 in the presence of SAP (FIG. 17B and FIG. 17C), it was assumed that recruitment of these complement regulatory factors inhibits the formation of TCC.

### 4-2. Evaluation of Complement Activation Late-Phase Reaction Using ELISA System

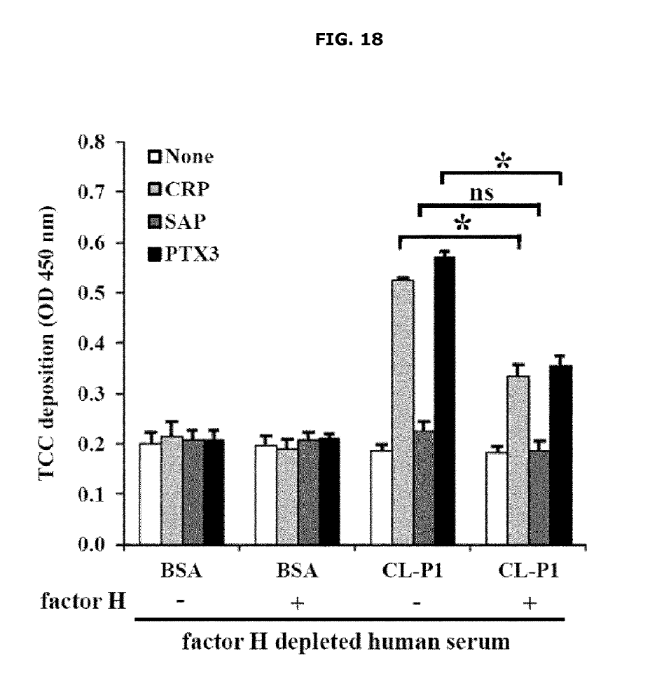

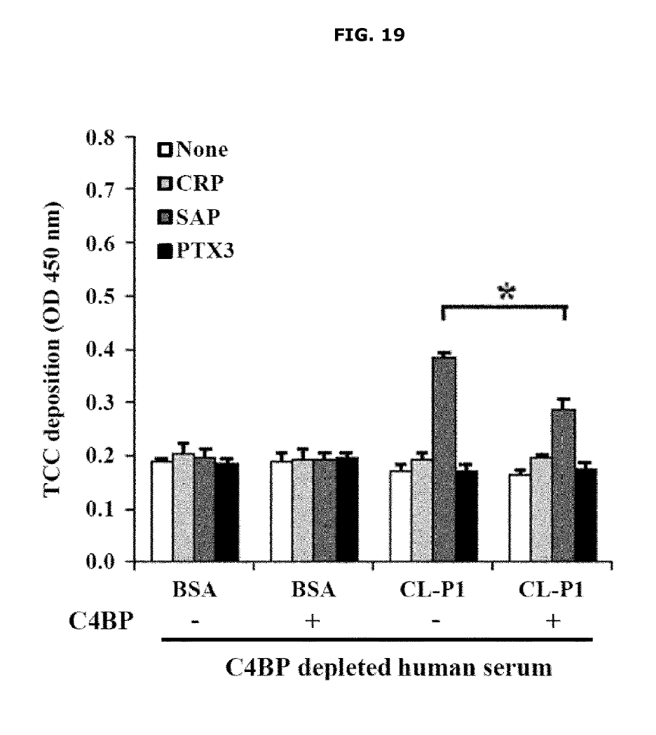

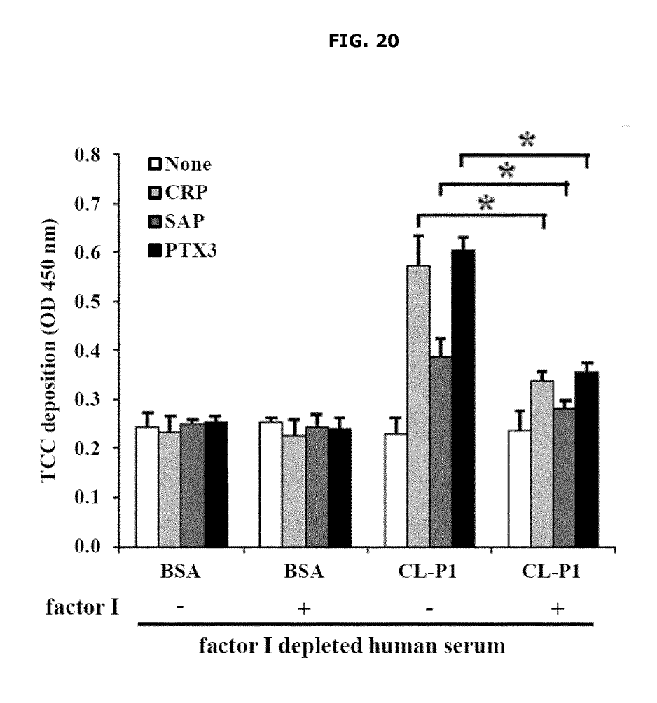

Induction of complement activation late-phase reaction by binding between CL-P1 extracellular domain and PTXs in the presence of complement regulatory factor depleted serum was evaluated by using ELISA system. When CFH depleted serum was used, the C5b-9 deposition amount in a CL-P1 extracellular domain-immobilized well increased by addition of CRP or PTX3, however, addition of SAP did not influence on the C5b-9 deposition amount (FIG. 18). When C4BP depleted serum was used, the C5b-9 deposition amount in a CL-P1 extracellular domain-immobilized well increased by addition of SAP, however, addition of CRP or PTX3 did not influence on the C5b-9 deposition amount (FIG. 19). When CFI depleted serum was used, the C5b-9 deposition amount in a CL-P1 extracellular domain-immobilized well increased by addition of CRP, SAP or PTX3 (FIG. 20). Further, when these depleted serums were supplemented with a corresponding one of CFH, C4BP, and CFI, increase in the C5b-9 deposition amount was suppressed in every case. Also in the content of soluble C5b-9 (SC5b-9) in the reaction supernatant, increase due to CFH depletion and suppression of increase by CFH supplement were observed likewise in the C5b-9 deposition amount (data not shown). These indicate that CRP, SAP and PTX3 can activate the complement activation late-phase reaction and induce TCC formation by binding to CL-P1 extracellular domain, and the TCC forming ability is inhibited in the presence of CFH for CRP and PTX3, in the presence of C4BP for SAP, and in the presence of CFI for CRP, SAP and PTX3.

### 4-3. Evaluation of Complement Activation Late-Phase Reaction Using Cell System

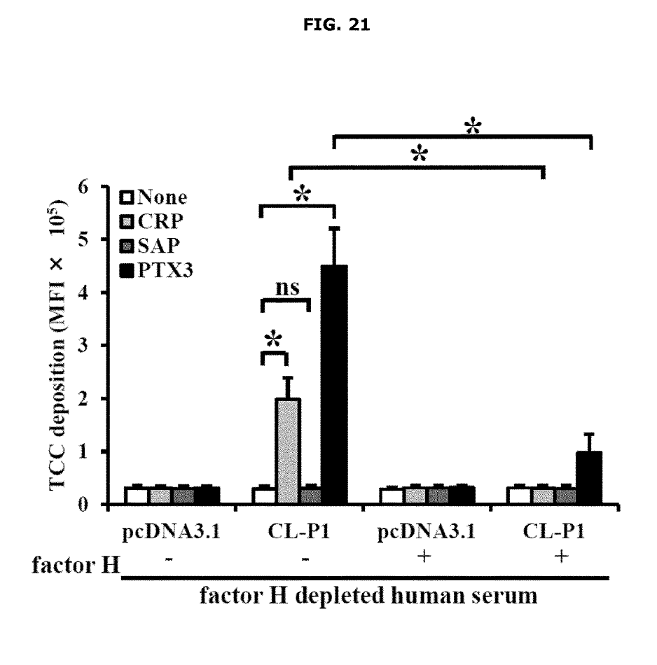

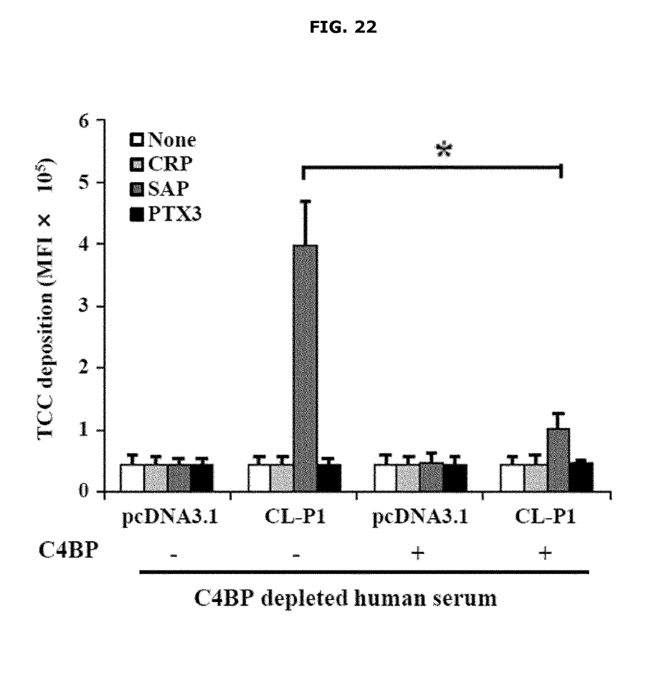

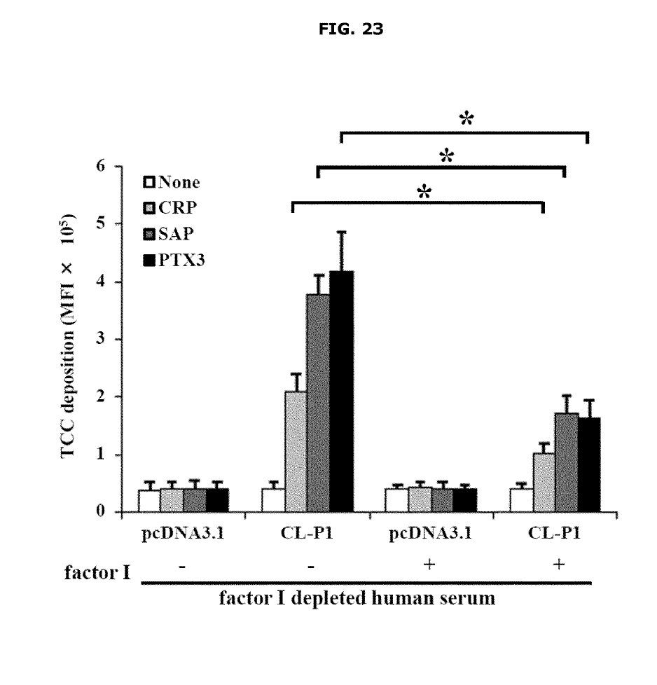

Induction of complement activation late-phase reaction by binding between CL-P1 and PTXs in the presence of complement regulatory factor depleted serum was evaluated by using CL-P1 expressing HEK293 cells. When CFH depleted serum was used, the C5b-9 deposition amount in CL-P1 expressing HEK293 cells increased by addition of CRP or PTX3, however, addition of SAP did not influence on the C5b-9 deposition amount (FIG. 21). When the C4BP depleted serum was used, the C5b-9 deposition amount in CL-P1 expressing HEK293 cells increased by addition of SAP, however, addition of CRP or PTX3 did not influence on the C5b-9 deposition amount (FIG. 22). When CFI depleted serum was used, the C5b-9 deposition amount in CL-P1 expressing HEK293 cells increased by addition of CRP, SAP or PTX3 (FIG. 23). Further, when a corresponding depleted serum was supplemented with CFH, C4BP, or CFI, increase in the C5b-9 deposition amount was suppressed in any case. These indicate that CRP, SAP and PTX3 can activate the complement activation late-phase reaction and induce TCC formation by binding to CL-P1 on the surface of HEK293 cells, and the TCC forming ability is inhibited in the presence of CFH for CRP and PTX3, in the presence of C4BP for SAP, and in the presence of CFI for CRP, SAP and PTX3

### Example 5 Evaluation of Complement Activation Late-Phase Reaction in aHUS Patient Serum Using Cell System

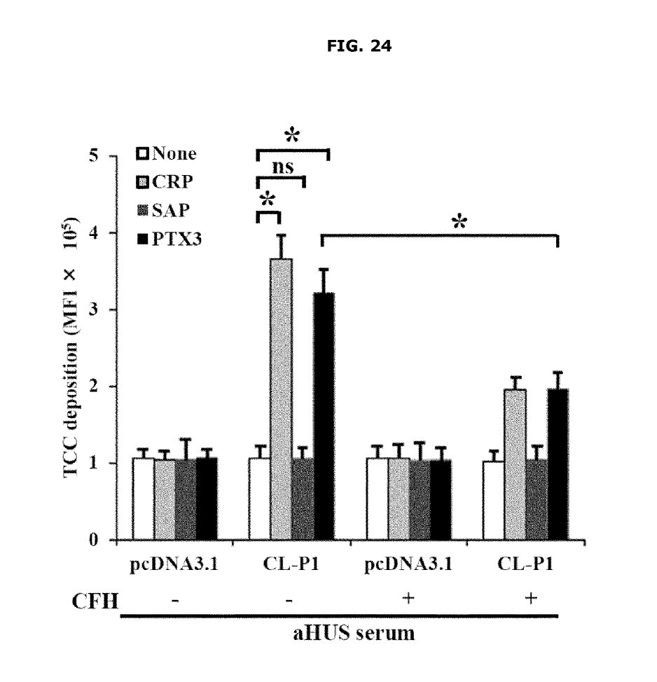

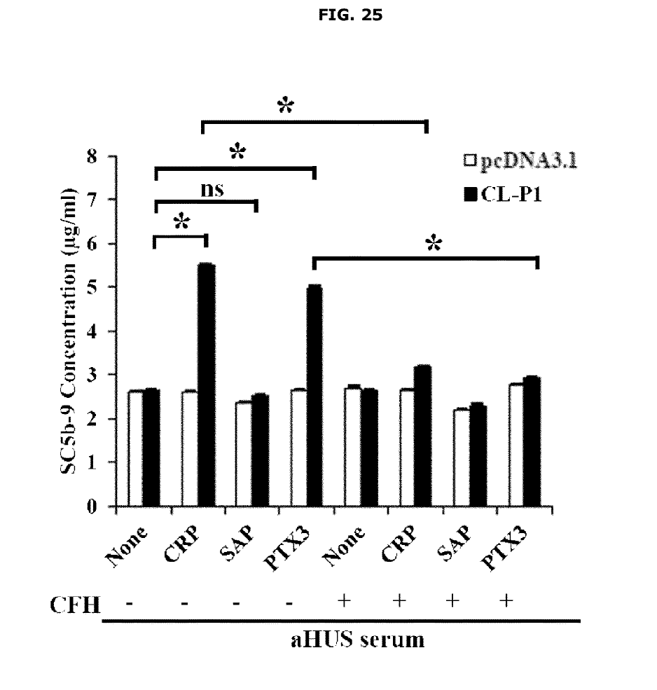

Using CL-P1 expressing HEK293 cells, complement activation late-phase reaction in aHUS patient serum was evaluated. In the presence of aHUS patient serum, the C5b-9 deposition amount in CL-P1 expressing HEK293 cells increased by addition of CRP or PTX3, and increase in the C5b-9 deposition amount was suppressed by supplement with CFH to aHUS patient serum (FIG. 24). Likewise the C5b-9 deposition amount, the SC5b-9 content in the reaction supernatant also increased by addition of CRP or PTX3, and the increase was suppressed by CFH supplement (FIG. 25). These reveal that in this aHUS patient serum, activation of the complement late-phase reaction by binding between CL-P1 and PTXs is not suppressed due to the failure in normal function of CFH, and thus TCC formation is enhanced.

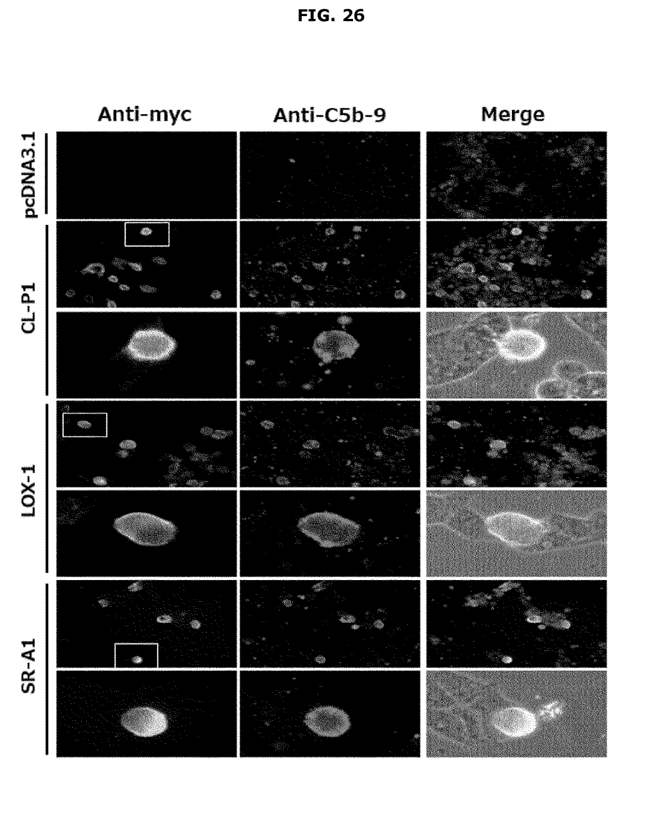

Next, using HEK293 cells expressing LOX-1 or SR-AI in place of CL-P1, complement activation late-phase reaction in the presence of CRP in aHUS patient serum was evaluated in the same manner. Also in the HEK293 cells expressing LOX-1 or SR-AI, increase in the C5b-9 deposition amount in the presence of CRP was observed as was observed in CL-P1 expressing HEK293 cells (FIG. 26), revealing that any of CL-P1, LOX-1, and SR-AI can be utilized in the complement reaction evaluation system.

### Example 6 Evaluation of Inhibition of Complement Activation Late-Phase Reaction by sCR1

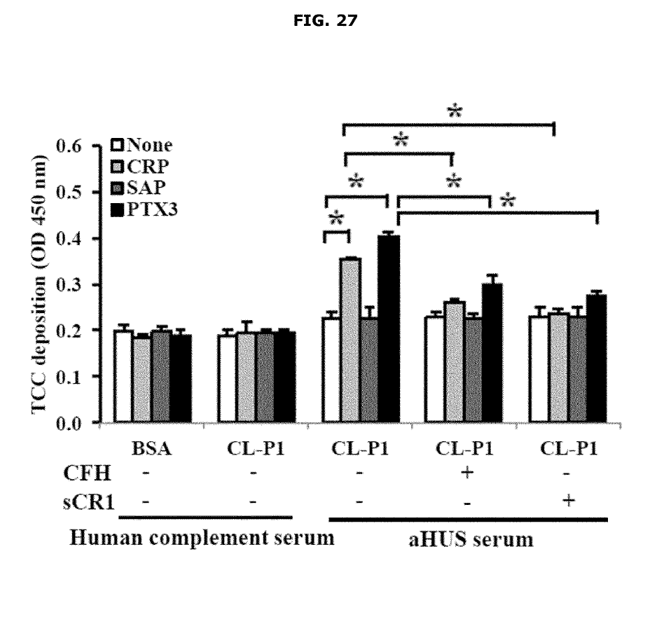

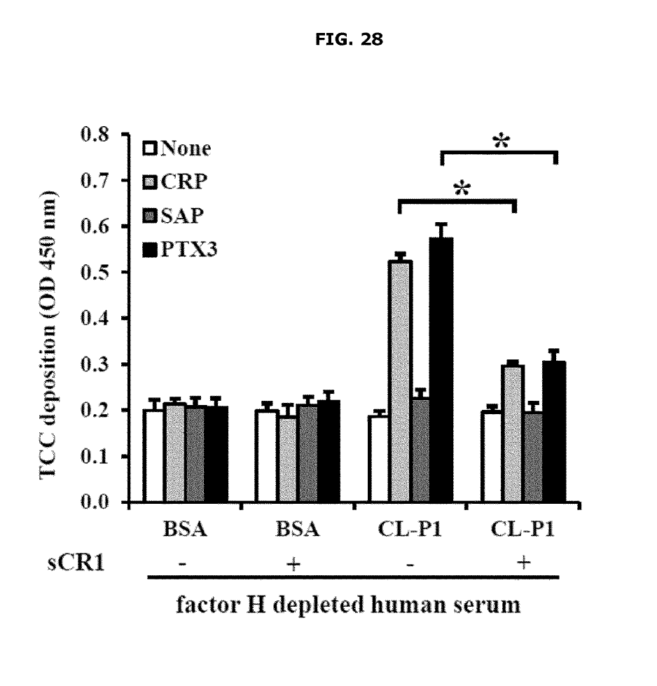

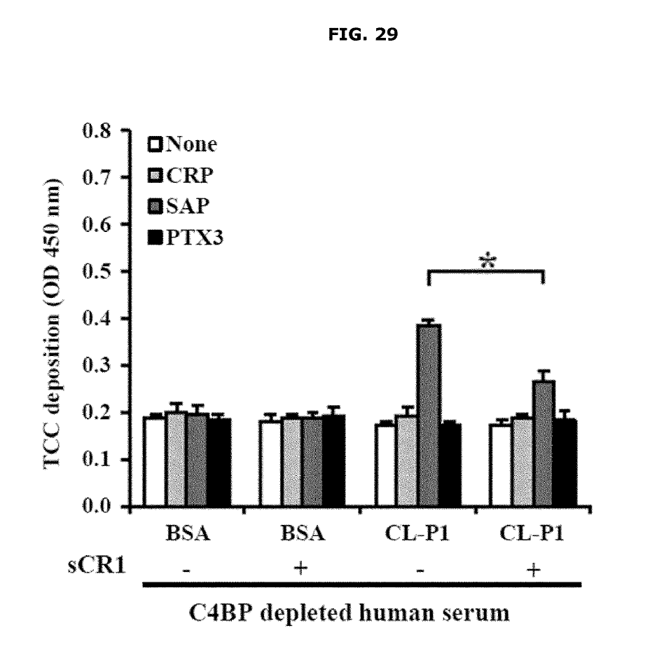

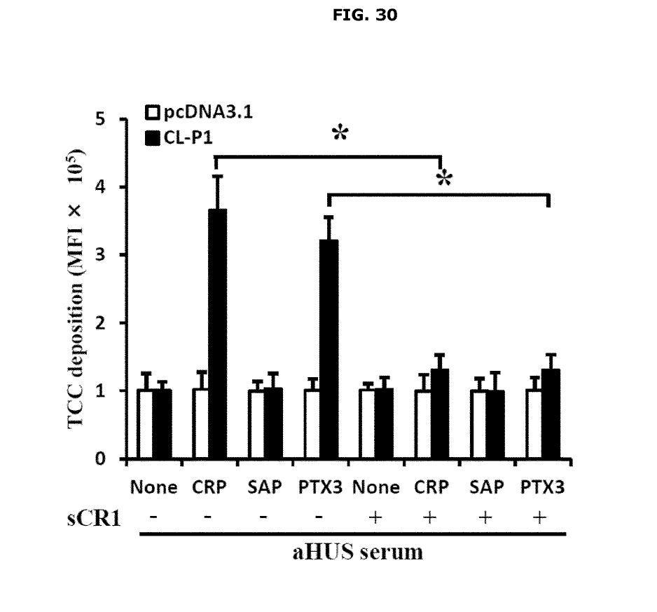

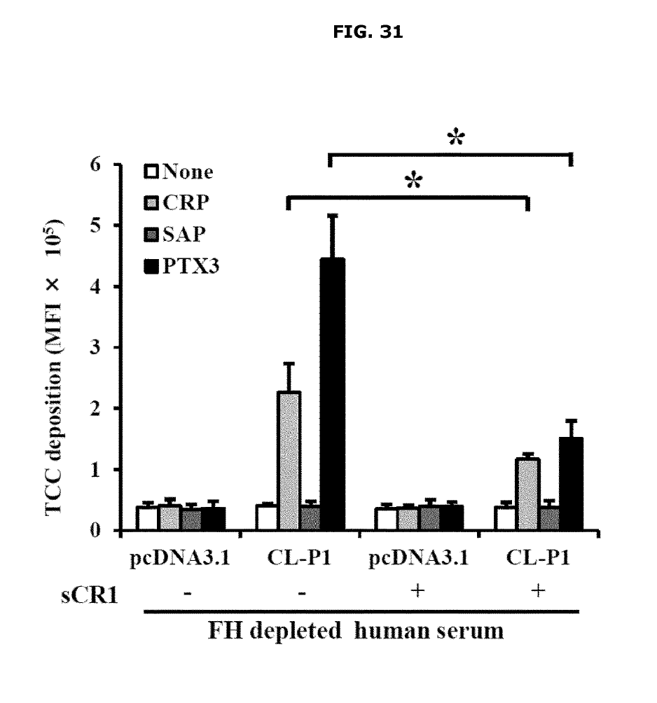

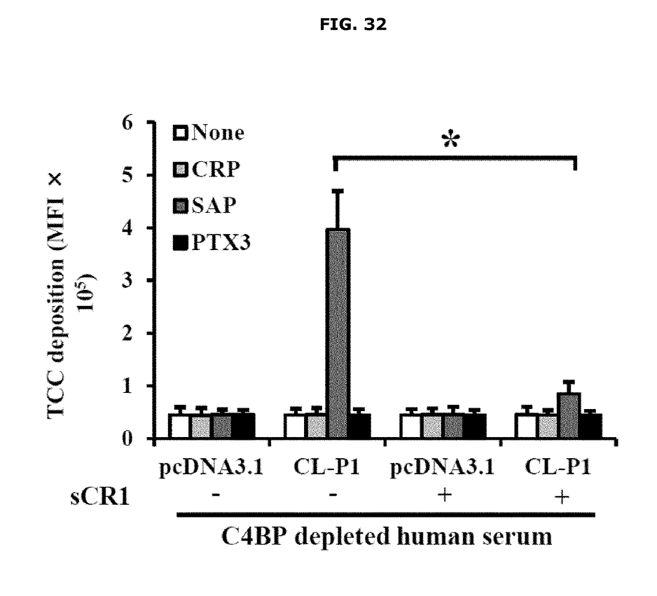

It is known that sCR1 inhibits complement activation late-phase reaction. Thus, it was evaluated if inhibition of complement activation late-phase reaction can be reproduced by adding sCR1 as an inhibitory substance to the ELISA system and the HEK293 cell system. In the ELISA system, the results of C5b-9 deposition amounts measured by using human complement serum, aHUS patient serum, CFH depleted serum, and C4BP depleted serum are shown in FIG. 27 to FIG. 29. Increase in the C5b-9 deposition amount in the presence of CRP or PTX3 at the time of addition of aHUS patient serum was suppressed also by addition of sCR1 as is the case with the addition of CFH (FIG. 27). Increase in the C5b-9 deposition amount in the presence of CRP or PTX3 at the time of addition of CFH depleted serum, and increase in the C5b-9 deposition amount in the presence of SAP at the time of addition of C4BP depleted serum were also suppressed by addition of sCR1 (FIG. 28, FIG. 29). Also, when various depleted serum or aHUS patient serum was used in the HEK293 cell system, increase in the C5b-9 deposition amount was suppressed by addition of sCR1 (FIG. 30 to FIG. 32). These confirmed that complement activation late-phase reaction is inhibited by sCR1 in any of the aforementioned ELISA systems and HEK293 cell systems.

These examples confirmed that any of the aforementioned ELISA systems and cell systems reflect the human complement system, and can be used for examining activation and/or abnormality of the complement system, and for screening the drugs that act on the complement system.

What is claimed is:

1. A method for examining a complement system of a subject, comprising:
a reaction step of letting a scavenger receptor or a functional equivalent thereof, a pentraxin family protein or a functional equivalent thereof and a blood sample collected from the subject coexist in vitro; and
a detection step of detecting complement activation amplifying reaction and/or complement activation late-phase reaction induced by interaction between the scavenger receptor or the functional equivalent thereof and the pentraxin family protein or the functional equivalent thereof from a reactant after the reaction step.

2. The method according to claim 1, wherein in the detection step, complement activation early-phase reaction is further detected.

3. The method according to claim 1, wherein the reaction step is a step of letting the scavenger receptor or the functional equivalent thereof existing on mammalian cells, the pentraxin family protein or the functional equivalent thereof and the blood sample collected from the subject coexist.

4. The method according to claim 3, wherein the mammalian cells are human cells.

5. The method according to claim 3, wherein the mammalian cells are human embryonic kidney-derived cells.

6. The method according to claim 1, wherein the reaction step is a step of letting the scavenger receptor or the functional equivalent thereof immobilized on a support, the pentraxin family protein or the functional equivalent thereof and the blood sample collected from the subject coexist.

7. The method according to claim 6, wherein the functional equivalent of the scavenger receptor is a peptide fragment containing an extracellular domain of the scavenger receptor.

8. The method according to claim 1, wherein the scavenger receptor is CL-P1, SR-AI or LOX-1.

9. The method according to claim 1, wherein the pentraxin family protein is C-reactive protein, serum amyloid P component or pentraxin 3.

10. The method according to claim 1, wherein the detection of the complement activation amplifying reaction is conducted by detection of properdin and/or factor B or a degradation product thereof.

11. The method according to claim 1, wherein the detection of the complement activation late-phase reaction is conducted by detection of C5b-9.

12. The method according to claim 2, wherein the detection of the complement activation early-phase reaction is conducted by detection of a C3 degradation product.

13. A kit for examining a complement system, comprising:
human cells expressing a scavenger receptor or a functional equivalent thereof; and
a pentraxin family protein or a functional equivalent thereof.

14. A method for testing a test substance for the ability to activate or the ability to suppress a complement system, comprising:
a test substance reaction step of letting a scavenger receptor or a functional equivalent thereof, a pentraxin family protein or a functional equivalent thereof, a blood sample and a test substance coexist in vitro;
a control reaction step of letting a scavenger receptor or a functional equivalent thereof, a pentraxin family protein or a functional equivalent thereof and a blood sample coexist in vitro;
a detection step of detecting complement activation amplifying reaction and/or complement activation late-phase reaction induced by interaction between the scavenger receptor or the functional equivalent thereof and the pentraxin family protein or the functional equivalent thereof from a reactant after each reaction step; and
a determination step of determining that the test substance has the ability to activate the complement system when the complement activation amplifying reaction and/or the complement activation late-phase reaction detected from the reactant after the test substance reaction step is larger than the complement activation amplifying reaction and/or the complement activation late-phase reaction detected from the reactant after the control reaction step, and determining that the test substance has the ability to suppress the complement system when the complement activation amplifying reaction and/or the complement activation late-phase reaction detected from the reactant after the test substance reaction step is smaller than the complement activation amplifying reaction and/or the complement activation late-phase reaction detected from the reactant after the control reaction step.
